(19) United States
(12) Patent Application Publication
(10) Pub. No.: US 2023/0365908 A1
(43) Pub. Date: Nov. 16, 2023

(54) DYNAMIC CELL CULTURE PLATFORM FOR COMBINATORIAL AND BIOMECHANICAL STIMULATION

(71) Applicant: UNIVERSITY OF UTAH RESEARCH FOUNDATION, Salt Lake City, UT (US)

(72) Inventors: Jungkyu Kim, Holladay, UT (US); Minju Kim, Salt Lake City, UT (US); Kanghoon Choi, Salt Lake City, UT (US); Zachary Ryan Estlack, Millcreek, UT (US)

(21) Appl. No.: 18/196,932

(22) Filed: May 12, 2023

Related U.S. Application Data
(60) Provisional application No. 63/341,629, filed on May 13, 2022.

(51) Int. Cl. C12M 1/00; C12M 3/06; C12M 1/34

(52) U.S. Cl. CPC C12M 23/26; C12M 23/16; C12M 29/14; C12M 41/40

(57) ABSTRACT

Described herein are apparatuses and methods for culturing and monitoring cells in dynamic physiological conditions with combinatorial and biomechanical stimulation. In some embodiments, the apparatuses and methods may comprise one or more cell culture chambers, flexible membranes, pneumatic actuators, microfluidic layers, or hydrogels. In some embodiments, various mechanical stimuli including bending stress, shear stress, or a combination thereof may be applied to one or more cell types. Also described herein are high-throughput and dynamic cell culture array systems comprising the described apparatuses and methods.

### CROSS-REFERENCE TO RELATED APPLICATION(S)

This application claims priority to U.S. Provisional Patent Application No. 63/341,629, filed on May 13, 2022, which is incorporated by reference herein in its entirety.

### BACKGROUND

Current dynamic culture systems are limited in that they do not have the ability to apply various biomechanical stimulations to mimic different biological environments. For example, the current biomimetic chip models for emulating dynamic motions only allow for unidirectional mechanical movement, which limits the ability to mimic the various physiological motions of more complex and non-flat organs. These current systems are also limited in their ability for high-throughput applications.

Thus, what is needed are new dynamic cell culture apparatuses and methods for reducing contamination, enabling the ability for various biomechanical stimulations, and increasing overall throughput. Such apparatuses and methods would be useful in a variety of cell culture applications and organ-chip models.

### SUMMARY

One embodiment described herein is a cell culture apparatus that may comprise: one or more cell culture chambers each comprising a flexible membrane, the flexible membrane separating an upper chamber and a pneumatic chamber and being mechanically integrated with the pneumatic chamber, wherein the pneumatic chamber is fluidly connected to a pneumatic actuator comprising a source of one or more pressurized fluids, the pneumatic actuator being configured to selectively adjust the pressure in the pneumatic chamber, thereby altering the shape of the flexible membrane through mechanical stimulation, and wherein the cell culture apparatus is configured to generate and apply various mechanical stimuli comprising bending stress, shear stress, or a combination thereof to one or more cell types. In one aspect, the flexible membrane may be comprised of a polymeric material comprising polycarbonate (PC), poly-methyl-meta-acrylate (PMMA), cyclic olefin copolymer (COC), polyimide, polydimethylsiloxane (PDMS), or combinations thereof. In another aspect, the flexible membrane may be comprised of PDMS. In another aspect, the flexible membrane may comprise a flat, concave, and/or convex shaped curvature upon mechanical stimulation to modulate bending stress on cells. In another aspect, the flexible membrane may comprise a uniform thickness across its surface of about 25 μm to about 250 μm. In another aspect, each of the one or more cell culture chambers may comprise an extracellular matrix layer comprising a hydrogel selected from the group consisting of collagen, elastin, alginate, and combinations thereof disposed on the flexible membrane. In another aspect, the apparatus may further comprise one or more perfusion channels, perfusion channel inlets, and perfusion channel outlets each fluidly connected to the hydrogel and configured to deliver one or more liquid fluids to the hydrogel. In another aspect, the flexible membrane may be comprised of one or more microfluidic layers each independently comprising one or more microfluidic chambers, microfluidic inlets, and microfluidic outlets disposed therein, each of the microfluidic chambers being fluidly connected through one or more porous membranes. In another aspect, at least one of the one or more microfluidic chambers may further comprise an extracellular matrix layer comprising a hydrogel selected from the group consisting of collagen, elastin, alginate, and combinations thereof. In another aspect, the apparatus may further comprise a syringe pump fluidly connected to at least one of the one or more microfluidic inlets or microfluidic outlets, the syringe pump comprising a source of one or more pressurized liquid fluids and configured to modulate shear stress on cells through the microfluidic chambers. In another aspect, the syringe pump may generate liquid fluid flow rates ranging from about 50 μL/sec to about 150 μL/sec through the microfluidic chambers. In another aspect, the one or more pressurized fluids of the pneumatic actuator may comprise air, liquid, or a combination thereof. In another aspect, the pneumatic actuator may generate fluid pressures ranging from about 5 kPa to about 50 kPa through the pneumatic chamber. In another aspect, the pneumatic actuator may generate fluid pressures at frequencies ranging from about 0.05 Hz to about 5 Hz through the pneumatic chamber. In another aspect, the pneumatic chamber may be fluidly connected to the pneumatic actuator through an interface component that may comprise: one or more interface inlets configured to receive the one or more pressurized fluids from the pneumatic actuator; one or more interface channels; one or more interface outlets configured to apply the one or more pressurized fluids to the pneumatic chamber; and one or more chamber portion outlets. In another aspect, the interface component may further comprise one or more apertures defining the one or more cell culture chambers. In another aspect, the apparatus may further comprise a base component connecting the one or more cell culture chambers to the interface component. In another aspect, the apparatus may comprise one or more pluralities of cell culture chambers each comprising multiple pneumatic chambers fluidly connected through one or more channels independent from other pluralities of cell culture chambers, and each comprising selectively adjusted pressures generated from the pneumatic actuator independent from other pluralities of cell culture chambers.

Another embodiment described herein is a method for culturing and monitoring one or more cell types in dynamic physiological conditions, the method may comprise: (a) inserting one or more cell types into a cell culture apparatus that may comprise: one or more cell culture chambers each comprising a flexible membrane, the flexible membrane separating an upper chamber and a pneumatic chamber and being mechanically integrated with the pneumatic chamber, wherein the pneumatic chamber is fluidly connected to a pneumatic actuator comprising a source of one or more pressurized fluids, the pneumatic actuator being configured to selectively adjust the pressure in the pneumatic chamber, thereby altering the shape of the flexible membrane through mechanical stimulation, and wherein the cell culture apparatus is configured to generate and apply various mechanical stimuli comprising bending stress, shear stress, or a combination thereof to one or more cell types; (b) applying one or more pressurized fluids to the pneumatic chamber using the pneumatic actuator, thereby altering the shape of the flexible membrane through mechanical stimulation and modulating bending stress on the cells; and (c) analyzing the cells in the cell culture apparatus. In one aspect, the flexible membrane may be comprised of one or more microfluidic layers each independently comprising one or more microfluidic chambers, microfluidic inlets, and microfluidic outlets disposed therein, each of the microfluidic chambers being fluidly connected through one or more porous membranes, and wherein the cell culture apparatus further comprises a syringe pump fluidly connected to at least one of the one or more microfluidic inlets or microfluidic outlets, the syringe pump comprising a source of one or more pressurized liquid fluids and configured to modulate shear stress on cells through the microfluidic chambers. In another aspect, the method may further comprise applying one or more pressurized liquid fluids to the one or more microfluidic inlets or microfluidic outlets using the syringe pump, thereby modulating shear stress on the cells through the microfluidic chambers. In another aspect, the syringe pump may generate liquid fluid flow rates ranging from about 50 μL/sec to about 150 μL/sec through the microfluidic chambers. In another aspect, at least one of the one or more microfluidic chambers may further comprise an extracellular matrix layer comprising a hydrogel selected from the group consisting of collagen, elastin, alginate, and combinations thereof. In another aspect, the one or more cell types may be cultured on the hydrogel, within the hydrogel, or a combination thereof. In another aspect, two or more different cell types may be co-cultured within the hydrogel. In another aspect, the cells may be analyzed in the cell culture apparatus using one or more imaging techniques. In another aspect, the method may further comprise performing one or more biochemical assays on the cells. In another aspect, the one or more pressurized fluids may be applied to the pneumatic chamber at one or more fluid pressures ranging from about 5 kPa to about 50 kPa. In another aspect, the one or more pressurized fluids may be applied to the pneumatic chamber at one or more frequencies ranging from about 0.05 Hz to about 5 Hz.

Another embodiment described herein is a method for culturing and monitoring one or more cell types in dynamic physiological conditions, the method may comprise: (a) inserting one or more cell types into a cell culture apparatus that may comprise: one or more cell culture chambers each comprising a flexible membrane, the flexible membrane separating an upper chamber and a pneumatic chamber and being mechanically integrated with the pneumatic chamber, wherein the pneumatic chamber is fluidly connected to a pneumatic actuator comprising a source of one or more pressurized fluids, the pneumatic actuator being configured to selectively adjust the pressure in the pneumatic chamber, thereby altering the shape of the flexible membrane through mechanical stimulation, wherein the cell culture apparatus is configured to generate and apply various mechanical stimuli comprising bending stress, shear stress, or a combination thereof to one or more cell types, and wherein each of the one or more cell culture chambers comprises an extracellular matrix layer comprising a hydrogel selected from the group consisting of collagen, elastin, alginate, and combinations thereof disposed on the flexible membrane; (b) applying one or more pressurized fluids to the pneumatic chamber using the pneumatic actuator, thereby altering the shape of the flexible membrane through mechanical stimulation and modulating bending stress on the cells; and (c) analyzing the cells in the cell culture apparatus. In one aspect, the one or more cell types may be cultured on the hydrogel, within the hydrogel, or a combination thereof. In another aspect, two or more different cell types may be co-cultured within the hydrogel. In another aspect, the cell culture apparatus may further comprise one or more perfusion channels, perfusion channel inlets, and perfusion channel outlets each fluidly connected to the hydrogel and configured to deliver one or more liquid fluids to the hydrogel.

Another embodiment described herein is a cell culture apparatus that may comprise: (a) one or more cell culture chambers each comprising a flexible membrane comprised of one or more microfluidic layers each independently comprising one or more microfluidic chambers, microfluidic inlets, and microfluidic outlets disposed therein, each of the microfluidic chambers being fluidly connected through one or more porous membranes; (b) an extracellular matrix layer disposed within each of the one or more cell culture chambers, the extracellular matrix layer comprising a hydrogel selected from the group consisting of collagen, elastin, alginate, and combinations thereof; (c) a means for bonding or injecting the hydrogel to the flexible membrane to form a three-dimensional (3D) cell culture environment; (d) one or more perfusion channels, perfusion channel inlets, and perfusion channel outlets formed around the hydrogel and configured to deliver one or more cell culture media to the hydrogel; (e) a means for removing cell culture media from at least one cell culture chamber to enable air exposure, thereby initiating cell differentiation; and (f) a means for exposing the hydrogel to various biomechanical stimuli.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-B show illustrations for a dynamic cell culture system for culturing various human tissue cells to generate different organ-chip models. Mechanical stimuli are applied to the in vitro system to mimic the biological environment. FIG. 1A shows example organ and tissue groups comprising heart valves to mimic the cyclic motion cycle; cornea having a hemisphere shape; round window membrane of inner ear to mimic vibrating motion, and alveoli to mimic respiratory motion, and different microfluidic membrane chip designs for different organs according to their anatomical structures. FIG. 1B shows that the physiological environment can be recreated through dynamic mechanical stimuli using the disclosed dynamic culture system.

FIG. 2 shows an illustration depicting an exemplary cell culture chamber embodiment (100) as described herein.

FIG. 3A shows an illustration depicting an exemplary cell culture chamber embodiment (200) as described herein. FIG. 3B shows an illustration depicting an exemplary embodiment comprising a thin flexible PDMS membrane having a collagen gel hydrogel layer disposed on top for culturing cells.

FIG. 4A shows an illustration depicting an exemplary cell culture chamber embodiment (300) as described herein. FIG. 4B shows an illustration depicting an exemplary embodiment comprising a cell culture chamber including perfusion chambers, a hydrogel, and PDMS and glass layers. Inlet 1 is for feeding in culture media and inlet 2 is for pneumatic control to form desired curvatures. FIG. 4C-D show an example array of multiple cell culture chambers having hydrogel and perfusion chambers as shown in FIG. 4A-B. FIG. 4D shows a close-up view of the hydrogel-based curvature array chip. Culture media can be fed into the perimeter perfusion chamber channels where the hydrogel makes contact.

FIG. 5 shows an illustration depicting an exemplary cell culture chamber embodiment (400) as described herein.

FIG. 6 shows an illustration depicting an exemplary cell culture chamber embodiment (500) as described herein.

FIG. 7A-E show schematic illustrations of an artificial cornea organ-chip model using the cell culture chamber apparatuses and methods as described herein. FIG. 7A shows the mechano-actuation system before applying curvature (i.e., flat configuration). FIG. 7B shows the mechano-actuation system applying curvature and bending stress to emulate the cornea shape using pneumatic fluid flow. FIG. 7C shows the construction of stroma with collagen type I using a self-assembled method. FIG. 7D shows epithelium by applying air lifting. FIG. 7E shows a schematic of the cornea model including three vertically stacked compartments with three different cell types.

FIG. 8A shows a schematic illustration of a pneumatical array balloon chip as described herein. FIG. 8B shows an example array chip having multiple cell culture chambers on a single plate system. FIG. 8C shows side view images of different membrane curvatures comparing flat, low, medium (i.e., “normal”), and high following mechanical stimulation through a pneumatic chamber. FIG. 8D shows finite element analysis (FEA) simulation plots estimating strain profiles for the different membrane curvatures shown in FIG. 8C.

FIG. 9A-C show an example of a cell culture apparatus comprising a flexible membrane having multiple microfluidic layers and chambers for culturing different cell types. In FIG. 9A, the red microfluidic layer comprises an epithelial cell chamber, the yellow microfluidic layer comprises a stromal cell chamber, and the blue microfluidic layer comprises an endothelial cell chamber, all separated and fluidly connected by porous membranes. Below the flexible membrane is a pneumatic layer comprising a pneumatic chamber for membrane stimulation. FIG. 9B shows an example cell culture apparatus organ-chip comprising a flexible membrane having the three different microfluidic layers and cell types as depicted in FIG. 9A. FIG. 9C shows a representative confocal microscopy image of a three-layered chip with immortalized human epithelial cells stained with CellTracker™ Green CMFDA Dye (top and bottom cell layers) and immortalized human keratocytes loaded with 10% GELMA hydrogel stained with CellTracker™ Deep Red Dye (middle cell layer).

FIG. 10A shows representative immunofluorescence images of keratocytes, fibroblasts, and myofibroblasts stained for ALDH3 and α-SMA phenotype markers. Scale bars: 50 μm. FIG. 10B shows representative immunofluorescence images of keratocytes, fibroblasts, and myofibroblasts stained for ALDH3, α-SMA, collagen type I, and F-actin phenotype markers under different curvatures of flat control, low, medium, and high using the cell culture apparatuses as described herein. Scale bars: 100 μm. FIG. 10C-D show immunofluorescence quantitative analysis of the relative expression of ALDH3, α-SMA, and vinculin (focal adhesion marker) in keratocytes, fibroblasts, and myofibroblasts prior to applying any membrane curvature through pneumatic actuation. FIG. 10E-J show immunofluorescence quantitative analysis of the relative expression of ALDH3, α-SMA, and collagen type I phenotype markers in keratocytes, fibroblasts, and myofibroblasts with flat control (C), low (L), medium (M), and high (H) membrane curvatures.

FIG. 11A shows immunofluorescence quantitative analysis of the relative expression of vinculin (focal adhesion marker) in keratocytes, fibroblasts, and myofibroblasts with flat control (C), low (L), medium (M), and high (H) membrane curvatures. FIG. 11B shows immunofluorescence quantitative analysis of the relative mean intensity of vinculin (focal adhesion marker) at specific cell loci (center, middle, or edge) in keratocytes, fibroblasts, and myofibroblasts with flat control, low, medium, and high membrane curvatures.

FIG. 12A-B show quantitative analysis of cell alignment/orientation depending on the specific direction (A, B, or C) using ImageJ processing. FIG. 12B shows graphs of the cell orientation by the location of curvature for each direction. S3_1=basal, lower; and S3_7=apical, higher.

FIG. 13A-C show immunofluorescence quantitative analysis of the relative expression of various phenotype markers in keratocytes (FIG. 13A), fibroblasts (FIG. 13B), and myofibroblasts (FIG. 13C) with flat control (C), low (L), medium (M), and high (H) membrane curvatures.

FIG. 14A-B show a heart valve organ-chip recapitulating biomechanical environment of a heart valve leaflet. FIG. 14A shows a schematic illustration of an aortic valve and leaflet. FIG. 14B shows a schematic illustration of the heart valve organ-chip where the top layer contains a microfluidic channel where valvular cells are cultured and a bottom layer for pneumatic control.

Using this valve chip, flexure/bending as well as fluidic shear stresses on valvular cells can be modulated.

FIG. 15A-C show a microfabricated heart valve organ-chip containing a thin microfluidic flexible membrane apparatus with a pneumatic chamber. FIG. 15A shows a top view and a side view of the heart valve organ-chip under pressure in the pneumatic chamber after filling a microchannel with dye. FIG. 15B shows a cross-sectional view of the heart valve organ-chip with a porous PDMS membrane in the microfluidic membrane. FIG. 15C shows a confocal microscopy image from a heart valve organ-chip cell culture of hydrogel-embedded valvular interstitial cells (VICs) (red) and valvular endothelial cells (VECs) (yellow) over a porous PDMS membrane (green).

FIG. 16 (top) shows a heart valve organ-chip as described herein recapitulating leaflet motion. FIG. 16 (bottom) shows a cross-sectional view of a fibrosa-mimicking layer with collagen-rich VIC-embedded hydrogel and a cross-sectional view of a ventricularis-mimicking layer with elastin-rich VIC-embedded hydrogel.

FIG. 17 shows an overview image of the pneumatically controlled heart valve organ-chip array for high-throughput applications.

FIG. 18A-B show an example illustration depicting a high-throughput (HT) organ-chip containing a cell culture chamber and pneumatic control ports to recapitulate topology and biomechanical motion of target organs for multiplexing investigation. FIG. 18A shows that with a 96-well HT organ-chip, various parameters (e.g., curvature, flexure, bending stress, shear stress, etc.) may be simultaneously or independently controlled to mimic micro-physiological environments precisely. FIG. 18B shows a side view of the HT organ-chip of FIG. 18A.

FIG. 19A-H show an exemplary cell culture apparatus array for high-throughput applications. FIG. 19A shows an exemplary interface component for a 12-well layout that connects the described cell culture chamber arrays to a pneumatic actuator by interfacing with the microfluidic chip. 1—Point to secure each individual microfluidic chip to a base and the interface against the chip itself. 2—Barb fittings for connections to outside pneumatic and liquid pressure sources. 3—Barb connections for interfacing with the microfluidic chip itself, sized to match a 96-well plate footprint. 4—Cutouts/apertures to allow access to each individual cell culture chamber/well by 96-well based equipment and for imaging applications. Two smaller cutouts, diagonally opposed, are used to allow for different cell seeding methods. 5—Matching barbed features on the other end of the interface to be used as an outlet during initial filling. FIG. 19B shows a side view image cut through the first set of wells showing the entire integrated apparatus with a microfluidic chip layer comprising a plurality of cell culture chambers supported by a top interface component and a bottom base component, where the plurality of cell culture chambers is connected to an external pneumatic actuator pressure source through the upper interface component. FIG. 19C-H show the high-throughput system in different states of assembly. FIGS. 19C and 19F show 12-well and 30-well plate compartment chip configurations, respectively. FIGS. 19D and 19G show the 12-well and 30-well plate compartment chips with an interface component mounted on top. FIGS. 19E and 19H show the 12-well and 30-well plate compartment chips with an interface component mounted on top and a bottom base component, where the chips are connected to an external pneumatic actuator pressure source through the upper interface component. The 12-well chip in FIG. 19C can be used in 12-, 24-, 36-, or 48-well configurations, and the 30-well chip in FIG. 19F can be used in 30- or 60-well configurations. Larger configurations of the 12-well and 30-well chips are also possible beyond these sizes. FIG. 20 shows a schematic illustration of an exemplary lung organ-chip model. FIG. 20 shows a cross-sectional view of a lung compartment chip. White PDMS is used to produce a cell culture chamber/well, clear PDMS is used to culture cells, and mechanical stimulation and motion are provided through inlets and outlets connected to a pneumatic actuator.

FIG. 21A-C show the height change of a flexible membrane balloon in a lung organ-chip model by adding 5 μL until 100 μL is reached. FIG. 21A shows the height of the balloon with 100 μL introduced. FIG. 21B shows a linear strain calculation on the submerged membrane. FIG. 21C shows a height by volume experimental graph for the lung organ-chip by introducing volumes from 0 to 100 μL to the balloons. Based on the experimental graph, 52 μL is added initially to mimic the alveolus and an additional 16 μL is repeatedly added and removed to mimic a linear strain of 4% during normal breathing.

FIG. 22 shows a mechanical motion test on the lung organ-chip without the well. The compartment chip was connected to a pneumatic actuator syringe pump using 1/16″ tubing and a 1 mL syringe to demonstrate the mechanical stretch motion.

FIG. 23A-B show FEA modeling and analysis using COMSOL Physics. FIG. 23A shows the total displacement showing the maximum height of the initial curvature at 0.53588 mm. FIG. 23B shows the height of the 4% linear strain from the initial curvature at 0.74077 mm.

FIG. 24A-C show a minimum cell culture media volume test for air-liquid interface formation. FIG. 24A shows that a volume of 30 μL showed the maximum air exposure area of 10.8736 mm² using ImageJ. A meniscus was formed due to the surface tension of water. FIG. 24B shows an experimental graph of the exposed area by minimum volume. FIG. 24C shows an illustration of meniscus formation in the cell culture chamber/well with the flexible membrane balloon.

FIG. 25 shows F-actin, Collagen Type 1, and Hoechst 33258 immunofluorescence staining for each case at the edge, 500 μm, 1000 μm, 1500 μm, and center of the flexible membrane balloon. Due to the mechanical stress on the cells, cell density was increased towards the edge, and cells were stretched between 500 μm and 1500 μm, where the strain was increased the most on the curvature. Scale bar: 50 μm.

FIG. 26 shows F-actin (red), ZO-1 (green), and Hoechst 33258 (blue) immunofluorescence staining in cultured A549 lung carcinoma epithelial cells at the edge of curvature and the middle of curvature of the flexible membrane balloon.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “microfluidic” refers to the behavior, precise control, and manipulation of fluids that are geometrically and dimensionally constrained to a small scale (typically sub-millimeter) at which surface forces dominate volumetric forces. A “microfluidic channel,” “microchannel,” “microfluidic chamber,” “channel,” or “flow channel” all generally refer to a micron-scale channel used for fluidically connecting various components of apparatuses, systems, and devices according to specific embodiments of the disclosed invention. A microchannel typically has a rectangular, e.g., square, or a rounded cross-section, with side and depth dimensions of between about 10 and 500 μm. Fluids flowing in the microchannels may exhibit microfluidic behavior. When used to refer to a microchannel within the cell culture apparatuses of the present invention, the terms “microchannel,” “microfluidic chamber,” and “channel” are used interchangeably. “Perfusion channel” or “perfusion chamber” generally denotes channels designed for passage of media, reagents, or other fluids or gels, and in some embodiments, cells. “Perfusion channel” is sometimes used to indicate any perfusion passages or structures that allow media to perfuse to a cell culture area.

“Microfluidic device” or “microfluidic apparatus” refer to a device or apparatus comprising at least one microchannel or microchamber having a cross-sectional dimension of less than 1 millimeter (typically between about 10 and 500 μm). In some embodiments of the present invention, described cell culture apparatuses may comprise flexible membranes comprised of one or more microfluidic layers, the microfluidic layers being fabricated to create one or more of microfluidic chambers, channels, reservoirs, reaction areas, inlets, or outlets by one or more processes of laser cutting, injection molding, die cutting, milling, press cutting, layer-by-layer fabrication, 3D printing, lithography, or combinations thereof. In one embodiment, each microfluidic layer may independently comprise one or more microfluidic chambers, microfluidic inlets, and microfluidic outlets disposed therein, each of the microfluidic chambers being fluidly connected through one or more porous membranes.

As used herein, “pneumatic” is used to describe a device or other external source containing or operated by air, liquid, and/or gas fluid under pressure. As used herein, “pneumatic actuator” refers to a device or other external source that converts the energy of compressed and pressurized air, liquid, and/or gas fluid into a mechanical stimulation and motion. In some embodiments of the present invention, described cell culture apparatuses may comprise a pneumatic chamber mechanically integrated with a flexible membrane, wherein the pneumatic chamber is fluidly connected to a pneumatic actuator comprising a source of one or more pressurized fluids, the pneumatic actuator being configured to selectively adjust the pressure in the pneumatic chamber, thereby altering the shape of the flexible membrane through mechanical stimulation. In some embodiments, the one or more pressurized fluids of the pneumatic actuator comprise air, liquid, or a combination thereof. In some embodiments, the pneumatic actuator generates fluid pressures ranging from about 5 kPa to about 50 kPa through the pneumatic chamber. In some embodiments, the pneumatic actuator generates fluid pressures at frequencies ranging from about 0.05 Hz to about 5 Hz through the pneumatic chamber.

As used herein, “polymer” or “polymeric material” is intended to encompass a homopolymer, heteropolymer, block polymer, co-polymer, ter-polymer, etc., and blends, combinations, or mixtures thereof. Examples of polymers may include, but are not limited to, functionalized polymers. Polymers include, without limitation, polyesters, poly(meth)acrylamides, poly(meth)acrylates, polyethers, polystyrenes, polynorbornenes and monomers that have unsaturated bonds. Examples of other polymers include, but are not limited to, polyalkylenes such as polyethylene and polypropylene; polychloroprene; polyvinyl ethers; such as polyvinyl acetate); polyvinyl halides such as polyvinyl chloride); polysiloxanes; polystyrenes; polyurethanes; polyacrylates; such as poly(methyl (meth)acrylate), poly(ethyl (meth)acrylate), poly(n-butyl(meth)acrylate), poly(isobutyl (meth)acrylate), poly(tert-butyl (meth)acrylate), poly(hexyl(meth)acrylate), poly(isodecyl (meth)acrylate), poly(lauryl (meth)acrylate), poly(phenyl (meth)acrylate), poly(methyl acrylate), poly(isopropyl acrylate), poly(isobutyl acrylate), and poly(octadecyl acrylate); polyacrylamides such as poly(acrylamide), poly(methacrylamide), poly(ethyl acrylamide), poly(ethyl methacrylamide), poly(N-isopropyl acrylamide), poly(n, iso, and tert-butyl acrylamide); and copolymers and mixtures thereof. These polymers may include useful derivatives, including polymers having substitutions, additions of chemical groups, for example, alkyl groups, alkylene groups, hydroxylations, oxidations, and other modifications routinely made by those skilled in the art. The polymers may include zwitterionic polymers such as, for example, polyphosphorycholine, polycarboxybetaine, and polysulfobetaine.

In some embodiments, cell culture apparatuses are described that may comprise one or more components comprised of hydrophilic polymeric material comprising one or more of polyacrylic acid, polymethylmethacrylate (PMMA), polycarbonate (PC), cyclic olefin copolymer (COC), polyimide, polydimethylsiloxane (PDMS), polyester, nylon, polyvinyl chloride, polyethylene, polypropylene, polyethylene terephthalate glycol, polybutylene adipate terephthalate, ethylene tetrafluoroethylene, fluorinated ethylene propylene, perfluoro alkoxy alkane, polylactic acid, polycaprolactone, polyoxymethylene, cellulose, co-polymers thereof, or combinations thereof. In one embodiment, a cell culture apparatus is described that comprises a flexible membrane comprised of a polymeric material comprising polycarbonate (PC), poly-methyl-meta-acrylate (PMMA), cyclic olefin copolymer (COC), polyimide, polydimethylsiloxane (PDMS), or combinations thereof. In another embodiment, the flexible membrane is comprised of PDMS.

Polymeric materials such as PDMS are typically hydrophobic. In certain embodiments of the disclosed invention, surface modification techniques including layer-by-layer (LBL) deposition, deposition of polyvinyl alcohol (PVA) following oxygen plasma bonding treatment, or poly(ethylene glycol) coating production can be performed on the polymeric material surfaces to finely control the hydrophilicity of the polymeric material and regulate overall fluid flow rates through the cell culture apparatus. In one embodiment, the modification may involve BSA deposition on the PDMS or on a substrate surface, such as glass. In another embodiment, the modification may involve oxygen plasma bonding treatment of the PDMS or the substrate surface. Plasma oxidation treatment renders the PDMS surface more hydrophilic, allowing aqueous solutions to maintain surface wetness. For applications where long-term hydrophilicity is necessary, techniques such as hydrophilic polymer grafting, surface nano-structuring, and dynamic surface modification with embedded surfactants can also be used.

As used herein, the term “organ-chip” refers to a cell culture apparatus comprising at least one cell type and physiological function of at least one mammalian (e.g., human) organ or tissue. While the organ-chips described herein mimic the physiological functions of a mammalian organ, it is to be understood that organ-chips can also be designed to mimic the functionality of any living organ from humans or other organisms (e.g., animals, insects, plants). As such, the apparatuses and methods described herein can be used to model or study mammalian as well as non-mammalian (e.g., insects, plants, etc.) organs and physiological systems and the effect of active agents and mechanical stimuli on such organs and physiological systems.

Organs are made up of different types of cells with various shaped backbone structures. To create realistic three-dimensional (3D) in vitro models, the diverse backbone structures are required to mimic the target organs and create organ-chips. Much effort has been devoted in the past to develop these 3D in vitro models. However, more investigations are needed in recapitulating biomechanical microenvironments associated with geometrical and dynamic aspects.

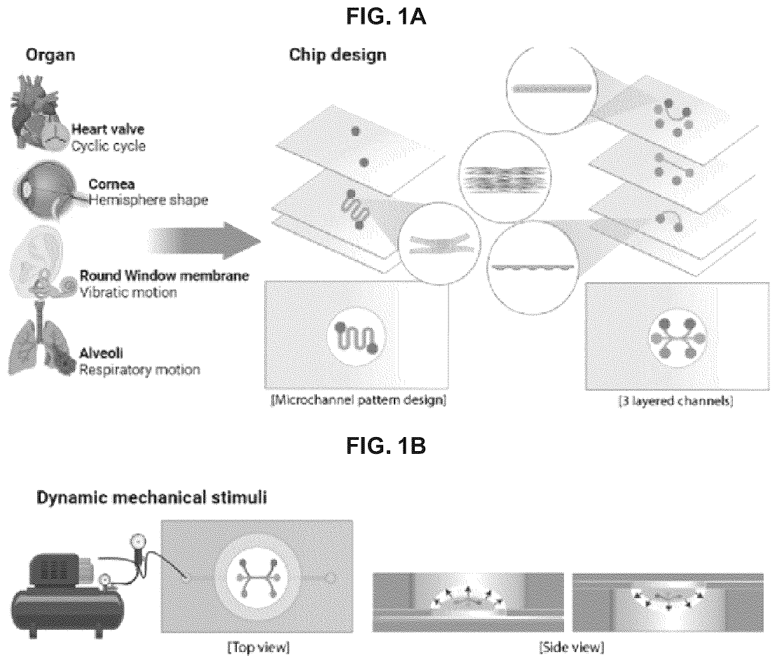

Described herein are cell culture apparatuses and methods that provide dynamic biomimetic microenvironments using various pneumatic and microfluidic control concepts to create different organ-chip models. FIG. 1A-B show illustrations for the disclosed dynamic cell culture systems for culturing various human tissue cells to generate different organ-chip models where mechanical stimuli are applied to the in vitro system to mimic the biological environment. FIG. 1A shows example organ and tissue groups that can be modeled using the described cell culture apparatuses and methods presented herein, and the different microfluidic membrane designs for different organs according to their anatomical structures. These organ and tissue groups include heart valves to mimic the cyclic motion cycle; cornea having a hemisphere shape; round window membrane of inner ear to mimic vibrating motion, and alveoli of the lung to mimic respiratory motion. FIG. 1B shows that the physiological environment can be recreated through dynamic mechanical stimuli (e.g., pneumatic actuation) using the disclosed dynamic culture systems.

The described cell culture apparatuses and methods solve all the previous issues of conventional dynamic cell culture systems by using a microfluidic cell culture chamber design comprising a thin and flexible membrane, which allows for shear and flexure/bending stresses to be dynamically applied to cells both independently and simultaneously. The described cell culture apparatuses and methods enable the reconstruction of precise geometrical aspects of organs such as concave or convex curvatures using controlled pneumatic actuation and mechanical stimulation. Thus, the disclosed invention aims to generate multiple biomechanical stimulations such as e.g., stretching, contraction, relaxation, and fluidic flow control under geometrically similar organ structures. By using the disclosed dynamic cell culture models, researchers can better predict the effects of drugs and other interventions on cellular behavior and function in vivo, improving the accuracy and reliability of preclinical studies.

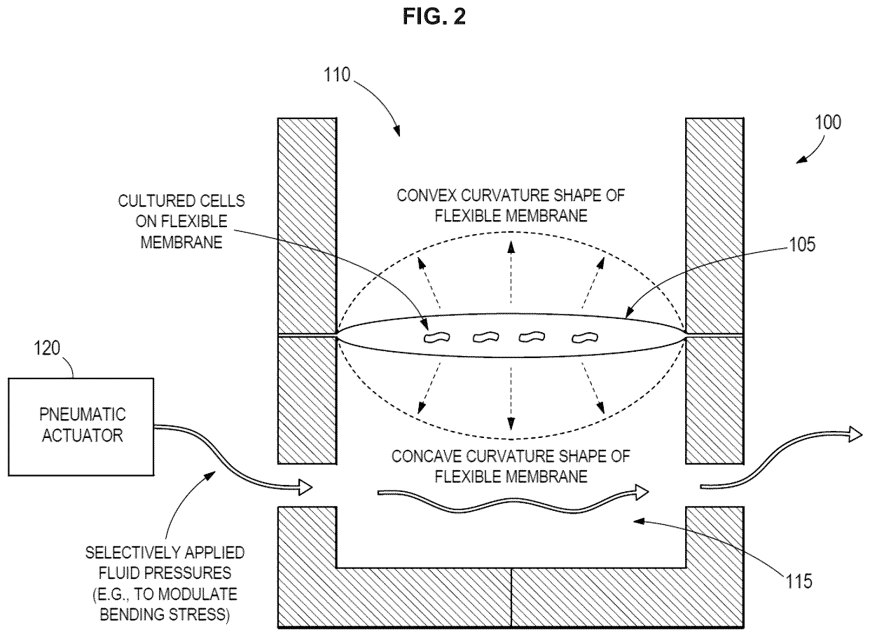

Described herein are cell culture apparatuses comprising one or more cell culture chambers. FIG. 2 shows a non-limiting exemplary embodiment of a cell culture chamber (100) comprising a flexible membrane (105) where one or more cell types are cultured. The flexible membrane (105) separates an upper chamber (110) and a lower pneumatic chamber (115), where the flexible membrane (105) is mechanically integrated with the pneumatic chamber (115). The pneumatic chamber (115) is fluidly connected to a pneumatic actuator (120) comprising a source of one or more pressurized fluids (e.g., air, liquid, or gas), where the pneumatic actuator (120) is configured to selectively apply and adjust fluid pressures in the pneumatic chamber (115), thereby altering the shape of the flexible membrane (105) through mechanical stimulation (e.g., to modulate bending stress and flexure). For example, the shape of the flexible membrane (105) may be modulated to comprise a flat, concave, and/or convex curvature upon mechanical stimulation to modulate bending stress on cells.

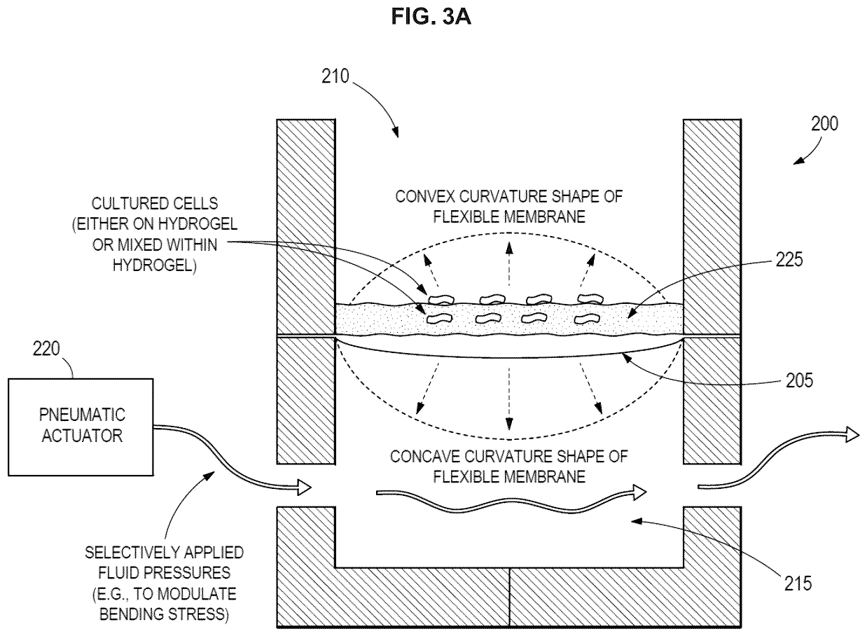

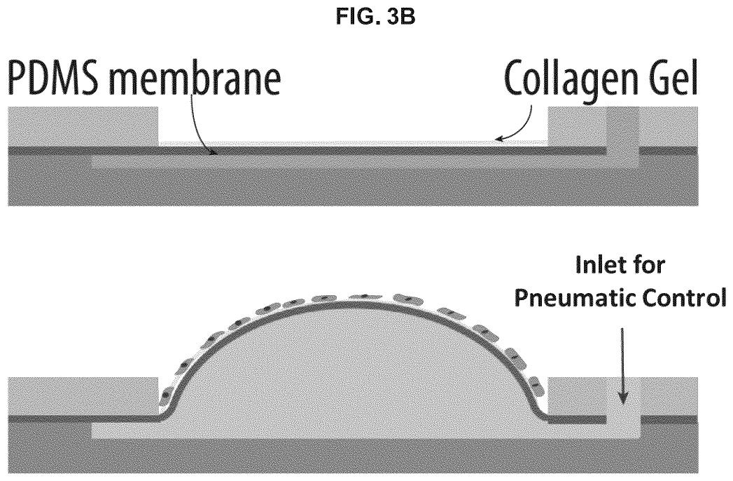

FIG. 3A shows a non-limiting exemplary embodiment of a cell culture chamber (200) comprising a flexible membrane (205) with a hydrogel (225) extracellular matrix layer disposed on top where one or more cell types are cultured. The one or more cell types may be cultured on the hydrogel (225), mixed within the hydrogel (225), or a combination thereof. In some embodiments, two or more different cell types may be co-cultured within the hydrogel (225). In some embodiments, the hydrogel (225) may be selected from collagen, elastin, alginate, a mixture thereof, or a combination thereof. Other suitable hydrogels known to those skilled in the art may also be used to mimic various 3D extracellular matrix environments. The flexible membrane (205) with the hydrogel (225) separates an upper chamber (210) and a lower pneumatic chamber (215), where the flexible membrane (205) is mechanically integrated with the pneumatic chamber (215). The pneumatic chamber (215) is fluidly connected to a pneumatic actuator (220) comprising a source of one or more pressurized fluids (e.g., air, liquid, or gas), where the pneumatic actuator (220) is configured to selectively apply and adjust fluid pressures in the pneumatic chamber (215), thereby altering the shape of the flexible membrane (205) and hydrogel (225) through mechanical stimulation (e.g., to modulate bending stress and flexure). For example, the shape of the flexible membrane (205) and hydrogel (225) may be modulated to comprise a flat, concave, and/or convex curvature upon mechanical stimulation to modulate bending stress on cells. FIG. 3B provides another schematic illustration of a cell culture chamber (200) exemplary embodiment comprising a collagen hydrogel layer disposed on top of a PDMS flexible membrane.

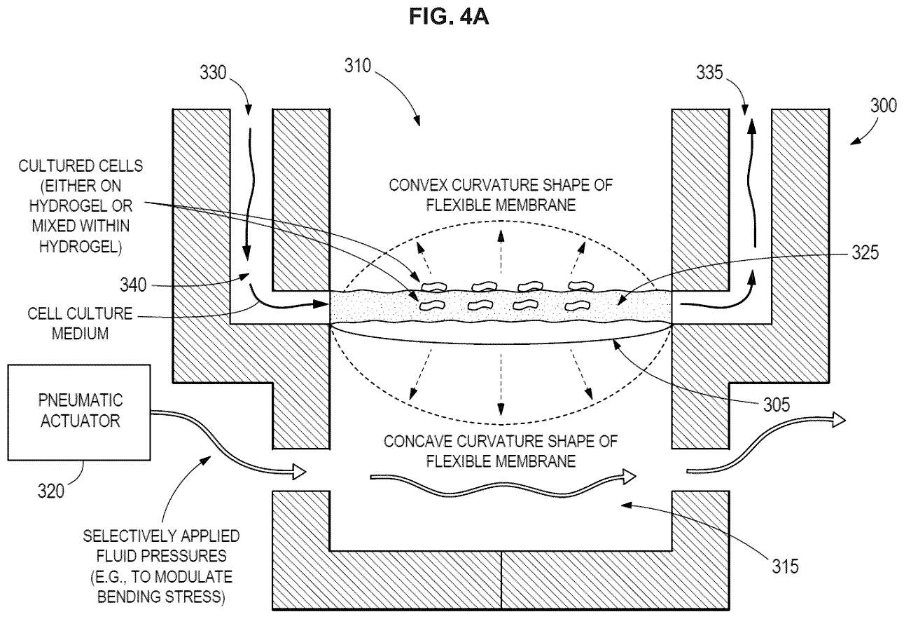

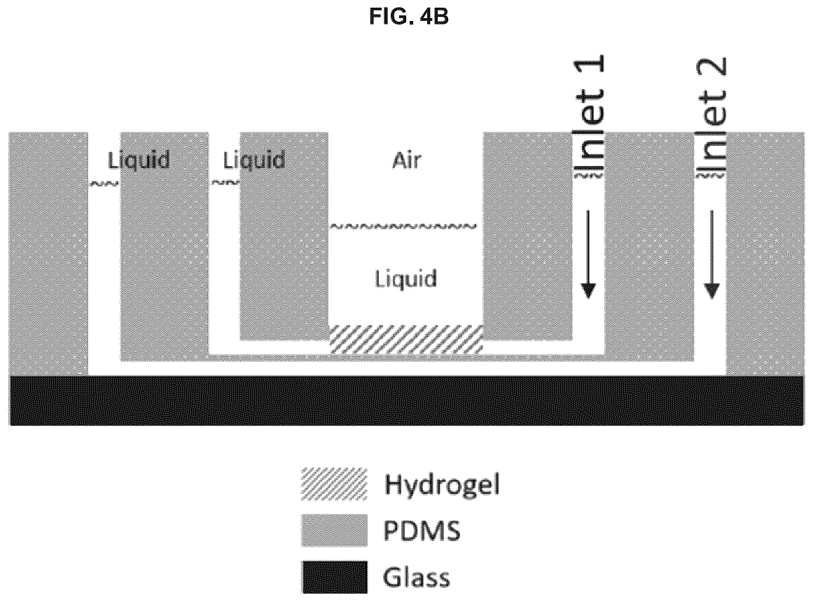

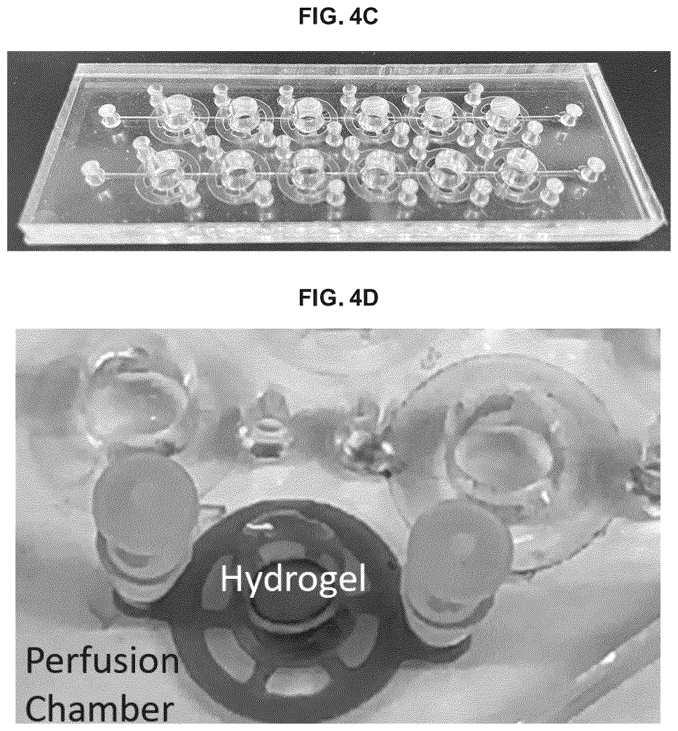

FIG. 4A shows a non-limiting exemplary embodiment of a cell culture chamber (300) comprising a flexible membrane (305) with a hydrogel (325) extracellular matrix layer disposed on top where one or more cell types are cultured. The cell culture chamber (300) further comprises one or more perfusion channel inlets (330), perfusion channels/chambers (340), and perfusion channel outlets (335) each fluidly connected to the hydrogel (325) and configured to deliver and supply one or more liquid fluids (e.g., cell culture medium) to the hydrogel (325) independently of the upper chamber (310). The one or more cell types may be cultured on the hydrogel (325), mixed within the hydrogel (325), or a combination thereof. In some embodiments, two or more different cell types may be co-cultured within the hydrogel (325). In some embodiments, the hydrogel (325) may be selected from collagen, elastin, alginate, a mixture thereof, or a combination thereof. Other suitable hydrogels known to those skilled in the art may also be used to mimic various 3D extracellular matrix environments. The flexible membrane (305) with the hydrogel (325) separates an upper chamber (310) and a lower pneumatic chamber (315), where the flexible membrane (305) is mechanically integrated with the pneumatic chamber (315). The pneumatic chamber (315) is fluidly connected to a pneumatic actuator (320) comprising a source of one or more pressurized fluids (e.g., air, liquid, or gas), where the pneumatic actuator (320) is configured to selectively apply and adjust fluid pressures in the pneumatic chamber (315), thereby altering the shape of the flexible membrane (305) and hydrogel (325) through mechanical stimulation (e.g., to modulate bending stress and flexure). For example, the shape of the flexible membrane (305) and hydrogel (325) may be modulated to comprise a flat, concave, and/or convex curvature upon mechanical stimulation to modulate bending stress on cells.

FIG. 4B provides another schematic illustration of a cell culture chamber (300) exemplary embodiment comprising a cell culture chamber including perfusion channels/chambers, a hydrogel, and PDMS and glass layers. Inlet 1 is for feeding in culture media to the hydrogel and inlet 2 is for pneumatic control to form different curvatures (flat, concave, and/or convex) of the flexible membrane and hydrogel. FIG. 4C-D show an example array configuration comprising multiple interconnected cell culture chambers having hydrogel and perfusion channels/chambers as shown in FIG. 4A-B. FIG. 4D shows a close-up view of the hydrogel-based curvature array chip. Culture media can be fed into the perimeter perfusion chamber channels where the hydrogel makes contact.

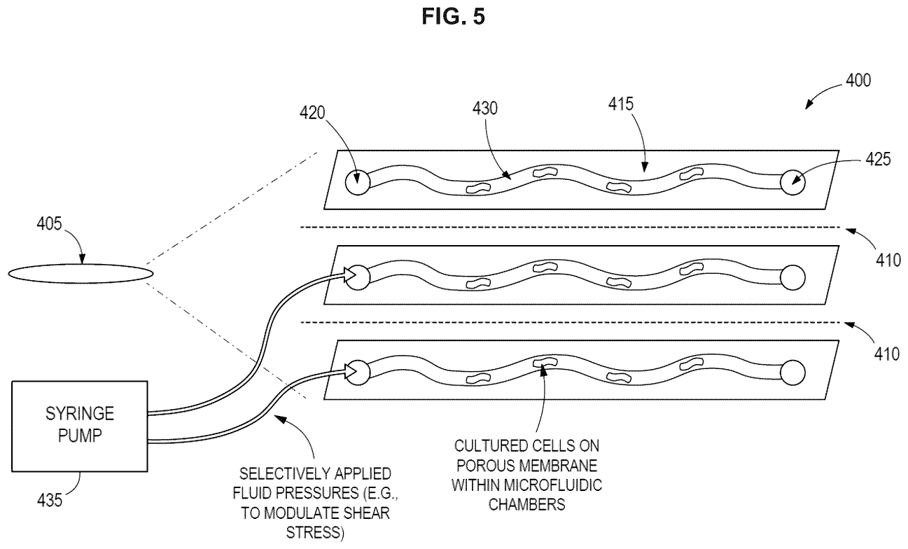

FIG. 5 shows a non-limiting exemplary embodiment of a microfluidic flexible membrane (400) that may be used in any of the cell culture chambers or apparatuses as described herein. A flexible membrane (405) is comprised of one or more microfluidic layers (415) each independently comprising one or more microfluidic inlets (420), microfluidic outlets (425), and microfluidic chambers (430) disposed therein, each of the microfluidic chambers being fluidly connected through one or more porous membranes (410). One or more cell types are cultured on the porous membranes (410) within microfluidic chambers (430) of the microfluidic layers (415). This embodiment comprises a syringe pump (435) that is fluidly connected to at least one of the one or more microfluidic inlets (420) and/or microfluidic outlets (425), where the syringe pump (435) comprises a source of one or more pressurized liquid fluids and is configured to modulate shear stress on cells through the microfluidic chambers (430). This allows for dynamic application of both shear and flexure/bending stresses both independently and simultaneously on cells.

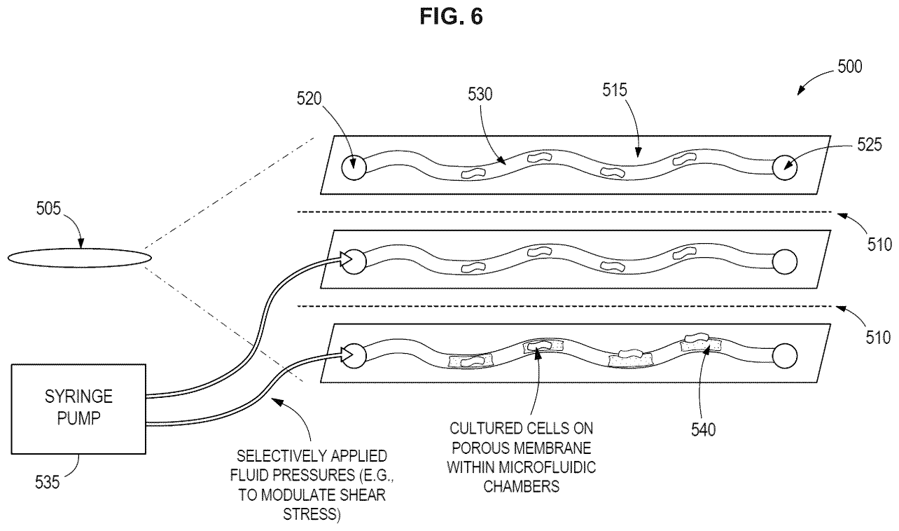

FIG. 6 shows a non-limiting exemplary embodiment of a microfluidic flexible membrane (500) comprising a hydrogel extracellular matrix layer that may be used in any of the cell culture chambers or apparatuses as described herein. A flexible membrane (505) is comprised of one or more microfluidic layers (515) each independently comprising one or more microfluidic inlets (520), microfluidic outlets (525), and microfluidic chambers (530) disposed therein, each of the microfluidic chambers being fluidly connected through one or more porous membranes (510). In this embodiment, one or more microfluidic chambers (530) comprise a hydrogel (540) extracellular matrix layer disposed therein for culturing one or more cell types on the porous membranes (510). The one or more cell types may be cultured on the hydrogel (540), mixed within the hydrogel (540), or a combination thereof. In some embodiments, two or more different cell types may be co-cultured within the hydrogel (540). In some embodiments, the hydrogel (540) may be selected from collagen, elastin, alginate, a mixture thereof, or a combination thereof. Other suitable hydrogels known to those skilled in the art may also be used to mimic various 3D extracellular matrix environments. This embodiment comprises a syringe pump (535) that is fluidly connected to at least one of the one or more microfluidic inlets (520) and/or microfluidic outlets (525), where the syringe pump (535) comprises a source of one or more pressurized liquid fluids and is configured to modulate shear stress on cells through the microfluidic chambers (530). This allows for dynamic application of both shear and flexure/bending stresses both independently and simultaneously on cells.

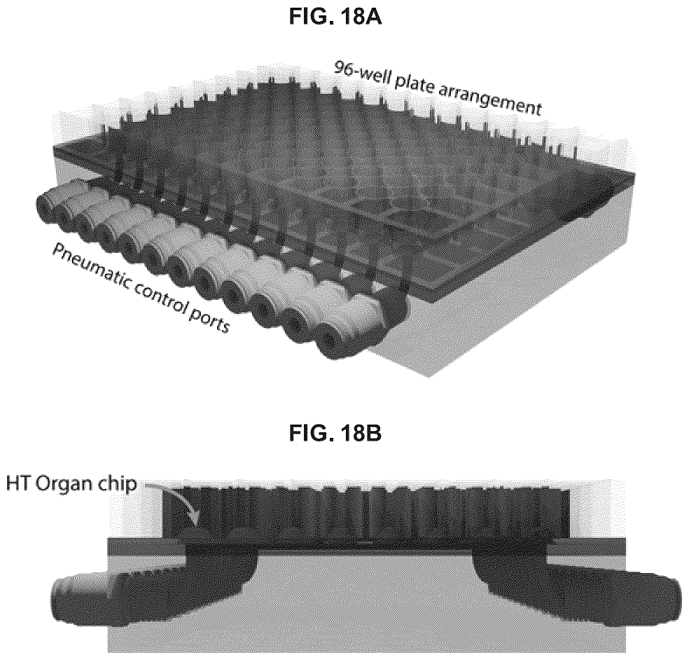

FIG. 18A-B show a non-limiting example cell culture apparatus illustration depicting a high-throughput (HT) 96-well array configuration comprising a plurality of cell culture chambers as described herein. The high-throughput format may comprise any one or any combination of the various cell culture chamber embodiments or flexible membrane embodiments as described herein to generate a high-throughput cell culture apparatus. FIG. 18A shows that with a 96-well HT organ-chip configuration and independent pneumatic control ports, various parameters (e.g., curvature, flexure, bending stress, shear stress, etc.) may be simultaneously or independently controlled to mimic microphysiological environments precisely. FIG. 18B shows a side view of the HT organ-chip of FIG. 18A.

In some embodiments of the present invention, described cell culture apparatuses may comprise one or more pluralities of cell culture chambers each comprising multiple pneumatic chambers fluidly connected through one or more channels independent from other pluralities of cell culture chambers, and each comprising selectively adjusted pressures generated from the pneumatic actuator independent from other pluralities of cell culture chambers. With these high-throughput organ-chip configurations, both static and dynamic cell cultures can be emulated to mimic different microenvironments such as those of the cornea, lung, heart valve, and inner ear to create specific organ-chips. The high-throughput organ-chip configurations also enable the rapid processing of large amounts of data or samples, potentially increasing the efficiency of in vitro drug screens. In addition, these array platforms can help to reduce human error and variability, resulting in more precise and consistent results. Furthermore, because high-throughput systems handle numerous samples at once, they can be less expensive than conventional, lower-throughput approaches. The high-throughput organ-chip platform can generate large datasets that, when combined with other data sources, lead to a better understanding of complex biological systems.

One embodiment described herein is a cell culture apparatus that may comprise: one or more cell culture chambers each comprising a flexible membrane, the flexible membrane separating an upper chamber and a pneumatic chamber and being mechanically integrated with the pneumatic chamber, wherein the pneumatic chamber is fluidly connected to a pneumatic actuator comprising a source of one or more pressurized fluids, the pneumatic actuator being configured to selectively adjust the pressure in the pneumatic chamber, thereby altering the shape of the flexible membrane through mechanical stimulation, and wherein the cell culture apparatus is configured to generate and apply various mechanical stimuli comprising bending stress, shear stress, or a combination thereof to one or more cell types. In one aspect, the flexible membrane may be comprised of a polymeric material comprising polycarbonate (PC), poly-methyl-meta-acrylate (PMMA), cyclic olefin copolymer (COC), polyimide, polydimethylsiloxane (PDMS), or combinations thereof. In another aspect, the flexible membrane may be comprised of PDMS. In another aspect, the flexible membrane may comprise a flat, concave, and/or convex shaped curvature upon mechanical stimulation to modulate bending stress on cells. In another aspect, the flexible membrane may comprise a uniform thickness across its surface of about 25 μm to about 250 μm. In another aspect, each of the one or more cell culture chambers may comprise an extracellular matrix layer comprising a hydrogel selected from the group consisting of collagen, elastin, alginate, and combinations thereof disposed on the flexible membrane. In another aspect, the apparatus may further comprise one or more perfusion channels, perfusion channel inlets, and perfusion channel outlets each fluidly connected to the hydrogel and configured to deliver one or more liquid fluids to the hydrogel. In another aspect, the flexible membrane may be comprised of one or more microfluidic layers each independently comprising one or more microfluidic chambers, microfluidic inlets, and microfluidic outlets disposed therein, each of the microfluidic chambers being fluidly connected through one or more porous membranes. In another aspect, at least one of the one or more microfluidic chambers may further comprise an extracellular matrix layer comprising a hydrogel selected from the group consisting of collagen, elastin, alginate, and combinations thereof. In another aspect, the apparatus may further comprise a syringe pump fluidly connected to at least one of the one or more microfluidic inlets or microfluidic outlets, the syringe pump comprising a source of one or more pressurized liquid fluids and configured to modulate shear stress on cells through the microfluidic chambers. In another aspect, the syringe pump may generate liquid fluid flow rates ranging from about 50 μL/sec to about 150 μL/sec through the microfluidic chambers. In another aspect, the one or more pressurized fluids of the pneumatic actuator may comprise air, liquid, or a combination thereof. In another aspect, the pneumatic actuator may generate fluid pressures ranging from about 5 kPa to about 50 kPa through the pneumatic chamber. In another aspect, the pneumatic actuator may generate fluid pressures at frequencies ranging from about 0.05 Hz to about 5 Hz through the pneumatic chamber. In another aspect, the pneumatic chamber may be fluidly connected to the pneumatic actuator through an interface component that may comprise: one or more interface inlets configured to receive the one or more pressurized fluids from the pneumatic actuator; one or more interface channels; one or more interface outlets configured to apply the one or more pressurized fluids to the pneumatic chamber; and one or more chamber portion outlets. In another aspect, the interface component may further comprise one or more apertures defining the one or more cell culture chambers. In another aspect, the apparatus may further comprise a base component connecting the one or more cell culture chambers to the interface component. In another aspect, the apparatus may comprise one or more pluralities of cell culture chambers each comprising multiple pneumatic chambers fluidly connected through one or more channels independent from other pluralities of cell culture chambers, and each comprising selectively adjusted pressures generated from the pneumatic actuator independent from other pluralities of cell culture chambers.

Another embodiment described herein is a method for culturing and monitoring one or more cell types in dynamic physiological conditions, the method may comprise: (a) inserting one or more cell types into a cell culture apparatus that may comprise: one or more cell culture chambers each comprising a flexible membrane, the flexible membrane separating an upper chamber and a pneumatic chamber and being mechanically integrated with the pneumatic chamber, wherein the pneumatic chamber is fluidly connected to a pneumatic actuator comprising a source of one or more pressurized fluids, the pneumatic actuator being configured to selectively adjust the pressure in the pneumatic chamber, thereby altering the shape of the flexible membrane through mechanical stimulation, and wherein the cell culture apparatus is configured to generate and apply various mechanical stimuli comprising bending stress, shear stress, or a combination thereof to one or more cell types; (b) applying one or more pressurized fluids to the pneumatic chamber using the pneumatic actuator, thereby altering the shape of the flexible membrane through mechanical stimulation and modulating bending stress on the cells; and (c) analyzing the cells in the cell culture apparatus. In one aspect, the flexible membrane may be comprised of one or more microfluidic layers each independently comprising one or more microfluidic chambers, microfluidic inlets, and microfluidic outlets disposed therein, each of the microfluidic chambers being fluidly connected through one or more porous membranes, and wherein the cell culture apparatus further comprises a syringe pump fluidly connected to at least one of the one or more microfluidic inlets or microfluidic outlets, the syringe pump comprising a source of one or more pressurized liquid fluids and configured to modulate shear stress on cells through the microfluidic chambers. In another aspect, the method may further comprise applying one or more pressurized liquid fluids to the one or more microfluidic inlets or microfluidic outlets using the syringe pump, thereby modulating shear stress on the cells through the microfluidic chambers. In another aspect, the syringe pump may generate liquid fluid flow rates ranging from about 50 μL/sec to about 150 μL/sec through the microfluidic chambers. In another aspect, at least one of the one or more microfluidic chambers may further comprise an extracellular matrix layer comprising a hydrogel selected from the group consisting of collagen, elastin, alginate, and combinations thereof. In another aspect, the one or more cell types may be cultured on the hydrogel, within the hydrogel, or a combination thereof. In another aspect, two or more different cell types may be co-cultured within the hydrogel. In another aspect, the cells may be analyzed in the cell culture apparatus using one or more imaging techniques. In another aspect, the method may further comprise performing one or more biochemical assays on the cells. Various cell assays may be performed directly on the disclosed cell culture apparatuses using standard optically based reagent kits and methods (e.g., fluorescence, absorbance, luminescence, etc.). For example, a cell viability assay utilizing conversion of a substrate to a fluorescent molecule by live cells may be performed. In addition, data can be collected directly on the cells/liquid in the cell culture apparatuses, such as by placing the cell culture apparatuses into a standard fluorescence plate reader. For cell imaging assays, the plate can be placed on a scanning microscope or high content system.

In another aspect, the one or more pressurized fluids may be applied to the pneumatic chamber at one or more fluid pressures ranging from about 5 kPa to about 50 kPa. In another aspect, the one or more pressurized fluids may be applied to the pneumatic chamber at one or more frequencies ranging from about 0.05 Hz to about 5 Hz.

Another embodiment described herein is a method for culturing and monitoring one or more cell types in dynamic physiological conditions, the method may comprise: (a) inserting one or more cell types into a cell culture apparatus that may comprise: one or more cell culture chambers each comprising a flexible membrane, the flexible membrane separating an upper chamber and a pneumatic chamber and being mechanically integrated with the pneumatic chamber, wherein the pneumatic chamber is fluidly connected to a pneumatic actuator comprising a source of one or more pressurized fluids, the pneumatic actuator being configured to selectively adjust the pressure in the pneumatic chamber, thereby altering the shape of the flexible membrane through mechanical stimulation, wherein the cell culture apparatus is configured to generate and apply various mechanical stimuli comprising bending stress, shear stress, or a combination thereof to one or more cell types, and wherein each of the one or more cell culture chambers comprises an extracellular matrix layer comprising a hydrogel selected from the group consisting of collagen, elastin, alginate, and combinations thereof disposed on the flexible membrane; (b) applying one or more pressurized fluids to the pneumatic chamber using the pneumatic actuator, thereby altering the shape of the flexible membrane through mechanical stimulation and modulating bending stress on the cells; and (c) analyzing the cells in the cell culture apparatus. In one aspect, the one or more cell types may be cultured on the hydrogel, within the hydrogel, or a combination thereof. In another aspect, two or more different cell types may be co-cultured within the hydrogel. In another aspect, the cell culture apparatus may further comprise one or more perfusion channels, perfusion channel inlets, and perfusion channel outlets each fluidly connected to the hydrogel and configured to deliver one or more liquid fluids to the hydrogel.

Another embodiment described herein is a cell culture apparatus that may comprise: (a) one or more cell culture chambers each comprising a flexible membrane comprised of one or more microfluidic layers each independently comprising one or more microfluidic chambers, microfluidic inlets, and microfluidic outlets disposed therein, each of the microfluidic chambers being fluidly connected through one or more porous membranes; (b) an extracellular matrix layer disposed within each of the one or more cell culture chambers, the extracellular matrix layer comprising a hydrogel selected from the group consisting of collagen, elastin, alginate, and combinations thereof; (c) a means for bonding or injecting the hydrogel to the flexible membrane to form a three-dimensional (3D) cell culture environment; (d) one or more perfusion channels, perfusion channel inlets, and perfusion channel outlets formed around the hydrogel and configured to deliver one or more cell culture media to the hydrogel; (e) a means for removing cell culture media from at least one cell culture chamber to enable air exposure, thereby initiating cell differentiation; and (f) a means for exposing the hydrogel to various biomechanical stimuli.

Another embodiment described herein is a cell culture apparatus that may be configured for compatibility with low throughput and high throughput processes and applications, including use with any multi-well configuration (e.g., 3-well, 12-well, 24-well, 30-well, 36-well, 48-well, 60-well, and 96-well plate configurations, and the like). In one aspect, the apparatus may comprise a means for generating concave and/or convex shaped curvatures in a cell culture environment under static and dynamic conditions. In another aspect, the apparatus may further comprise a means for modulating the frequency of said dynamic conditions.

Another embodiment described herein is a modular cell culture system for applying various mechanical stimuli to one or more cell samples, wherein said mechanical stimuli may be selectively applied to all cell samples or a subgroup thereof, thereby enabling maximization of the number of cell samples to be examined.

Another embodiment described herein is a cell culture apparatus that may comprise a flexible membrane having a thickness gradient, wherein the flexible membrane may be configured to generate different curvatures when subjected to a pressure source. In one aspect, the cell culture apparatus may further comprise a means for producing shear stress on one or more cell types by controlling a fluid flow rate across said flexible membrane. In another aspect, the flexible membrane structure may be comprised of one or more thin microfluidic channel layers in the cell culture apparatus, facilitating the creation of coupled or decoupled biomechanical stimuli, including but not limited to bending stress, flexure stress, and shear stress.

Another embodiment described herein is a functional corneal tissue organ-chip model system comprising a cell culture apparatus as described herein, wherein said apparatus supports static curvature formation.

Another embodiment described herein is a functional heart valve leaflet organ-chip model system comprising a cell culture apparatus as described herein, wherein said apparatus supports dynamic positive and negative leaflet formation.

Another embodiment described herein is a functional lung tissue organ-chip model system comprising a cell culture apparatus as described herein, wherein said apparatus supports dynamic curvature formation with frequency modulation. In one aspect, the cell culture apparatus may further comprise a curvature organ-chip including a hydrogel component.

Another embodiment described herein is a method for enhancing the overall mixing rate in an enzyme-linked immunosorbent assay (ELISA) or other biochemical assay. In one aspect, the method may comprise the use of an actuation mechanism derived from a cell culture apparatus as described herein. For example, the actuation of cell culture chambers can be used to induce fluid motion in culture wells, which can aid in mixing and increase reaction speeds to shorten assay times.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A cell culture apparatus comprising:
   one or more cell culture chambers each comprising a flexible membrane, the flexible membrane separating an upper chamber and a pneumatic chamber and being mechanically integrated with the pneumatic chamber, wherein the pneumatic chamber is fluidly connected to a pneumatic actuator comprising a source of one or more pressurized fluids, the pneumatic actuator being configured to selectively adjust the pressure in the pneumatic chamber, thereby altering the shape of the flexible membrane through mechanical stimulation, and wherein the cell culture apparatus is configured to generate and apply various mechanical stimuli comprising bending stress, shear stress, or a combination thereof to one or more cell types.
Clause 2. The apparatus of clause 1, wherein the flexible membrane is comprised of a polymeric material comprising polycarbonate (PC), poly-methyl-meta-acrylate (PMMA), cyclic olefin copolymer (COC), polyimide, polydimethylsiloxane (PDMS), or combinations thereof.
Clause 3. The apparatus of clause 1 or 2, wherein the flexible membrane is comprised of PDMS.
Clause 4. The apparatus of any one of clauses 1-3, wherein the flexible membrane comprises a flat, concave, and/or convex shaped curvature upon mechanical stimulation to modulate bending stress on cells.
Clause 5. The apparatus of any one of clauses 1-4, wherein the flexible membrane comprises a uniform thickness across its surface of about 25 μm to about 250 μm.
Clause 6. The apparatus of any one of clauses 1-5, wherein each of the one or more cell culture chambers comprises an extracellular matrix layer comprising a hydrogel selected from the group consisting of collagen, elastin, alginate, and combinations thereof disposed on the flexible membrane.
Clause 7. The apparatus of any one of clauses 1-6, further comprising one or more perfusion channels, perfusion channel inlets, and perfusion channel outlets each fluidly connected to the hydrogel and configured to deliver one or more liquid fluids to the hydrogel.
Clause 8. The apparatus of any one of clauses 1-7, wherein the flexible membrane is comprised of one or more microfluidic layers each independently comprising one or more microfluidic chambers, microfluidic inlets, and microfluidic outlets disposed therein, each of the microfluidic chambers being fluidly connected through one or more porous membranes.
Clause 9. The apparatus of any one of clauses 1-8, wherein at least one of the one or more microfluidic chambers further comprises an extracellular matrix layer comprising a hydrogel selected from the group consisting of collagen, elastin, alginate, and combinations thereof.
Clause 10. The apparatus of any one of clauses 1-9, further comprising a syringe pump fluidly connected to at least one of the one or more microfluidic inlets or microfluidic outlets, the syringe pump comprising a source of one or more pressurized liquid fluids and configured to modulate shear stress on cells through the microfluidic chambers.
Clause 11. The apparatus of any one of clauses 1-10, wherein the syringe pump generates liquid fluid flow rates ranging from about 50 μL/sec to about 150 μL/sec through the microfluidic chambers.
Clause 12. The apparatus of any one of clauses 1-11, wherein the one or more pressurized fluids of the pneumatic actuator comprise air, liquid, or a combination thereof.
Clause 13. The apparatus of any one of clauses 1-12, wherein the pneumatic actuator generates fluid pressures ranging from about 5 kPa to about 50 kPa through the pneumatic chamber.
Clause 14. The apparatus of any one of clauses 1-13, wherein the pneumatic actuator generates fluid pressures at frequencies ranging from about 0.05 Hz to about 5 Hz through the pneumatic chamber.
Clause 15. The apparatus of any one of clauses 1-14, wherein the pneumatic chamber is fluidly connected to the pneumatic actuator through an interface component comprising:
   one or more interface inlets configured to receive the one or more pressurized fluids from the pneumatic actuator;
   one or more interface channels;
   one or more interface outlets configured to apply the one or more pressurized fluids to the pneumatic chamber; and
   one or more chamber portion outlets.
Clause 16. The apparatus of any one of clauses 1-15, wherein the interface component further comprises one or more apertures defining the one or more cell culture chambers.
Clause 17. The apparatus of any one of clauses 1-16, further comprising a base component connecting the one or more cell culture chambers to the interface component.
Clause 18. The apparatus of any one of clauses 1-17, wherein the apparatus comprises one or more pluralities of cell culture chambers each comprising multiple pneumatic chambers fluidly connected through one or more channels independent from other pluralities of cell culture chambers, and each comprising selectively adjusted pressures generated from the pneumatic actuator independent from other pluralities of cell culture chambers.
Clause 19. A method for culturing and monitoring one or more cell types in dynamic physiological conditions, the method comprising:
   (a) inserting one or more cell types into a cell culture apparatus comprising:
      one or more cell culture chambers each comprising a flexible membrane, the flexible membrane separating an upper chamber and a pneumatic chamber and being mechanically integrated with the pneumatic chamber, wherein the pneumatic chamber is fluidly connected to a pneumatic actuator comprising a source of one or more pressurized fluids, the pneumatic actuator being configured to selectively adjust the pressure in the pneumatic chamber, thereby altering the shape of the flexible membrane through mechanical stimulation, and wherein the cell culture apparatus is configured to generate and apply various mechanical stimuli comprising bending stress, shear stress, or a combination thereof to one or more cell types;
   (b) applying one or more pressurized fluids to the pneumatic chamber using the pneumatic actuator, thereby altering the shape of the flexible membrane through mechanical stimulation and modulating bending stress on the cells; and
   (c) analyzing the cells in the cell culture apparatus.
Clause 20. The method of clause 19, wherein the flexible membrane is comprised of one or more microfluidic layers each independently comprising one or more microfluidic chambers, microfluidic inlets, and microfluidic outlets disposed therein, each of the microfluidic chambers being fluidly connected through one or more porous membranes, and wherein the cell culture apparatus further comprises a syringe pump fluidly connected to at least one of the one or more microfluidic inlets or microfluidic outlets, the syringe pump comprising a source of one or more pressurized liquid fluids and configured to modulate shear stress on cells through the microfluidic chambers.
Clause 21. The method of clause 19 or 20, further comprising applying one or more pressurized liquid fluids to the one or more microfluidic inlets or microfluidic outlets using the syringe pump, thereby modulating shear stress on the cells through the microfluidic chambers.
Clause 22. The method of any one of clauses 19-21, wherein the syringe pump generates liquid fluid flow rates ranging from about 50 μL/sec to about 150 μL/sec through the microfluidic chambers.
Clause 23. The method of any one of clauses 19-22, wherein at least one of the one or more microfluidic chambers further comprises an extracellular matrix layer comprising a hydrogel selected from the group consisting of collagen, elastin, alginate, and combinations thereof.
Clause 24. The method of any one of clauses 19-23, wherein the one or more cell types are cultured on the hydrogel, within the hydrogel, or a combination thereof.
Clause 25. The method of any one of clauses 19-24, wherein two or more different cell types are co-cultured within the hydrogel.
Clause 26. The method of any one of clauses 19-25, wherein the cells are analyzed in the cell culture apparatus using one or more imaging techniques.
Clause 27. The method of any one of clauses 19-26, further comprising performing one or more biochemical assays on the cells.
Clause 28. The method of any one of clauses 19-27, wherein the one or more pressurized fluids are applied to the pneumatic chamber at one or more fluid pressures ranging from about 5 kPa to about 50 kPa.
Clause 29. The method of any one of clauses 19-28, wherein the one or more pressurized fluids are applied to the pneumatic chamber at one or more frequencies ranging from about 0.05 Hz to about 5 Hz.
Clause 30. A method for culturing and monitoring one or more cell types in dynamic physiological conditions, the method comprising:
   (a) inserting one or more cell types into a cell culture apparatus comprising:
      one or more cell culture chambers each comprising a flexible membrane, the flexible membrane separating an upper chamber and a pneumatic chamber and being mechanically integrated with the pneumatic chamber, wherein the pneumatic chamber is fluidly connected to a pneumatic actuator comprising a source of one or more pressurized fluids, the pneumatic actuator being configured to selectively adjust the pressure in the pneumatic chamber, thereby altering the shape of the flexible membrane through mechanical stimulation, wherein the cell culture apparatus is configured to generate and apply various mechanical stimuli comprising bending stress, shear stress, or a combination thereof to one or more cell types, and wherein each of the one or more cell culture chambers comprises an extracellular matrix layer comprising a hydrogel selected from the group consisting of collagen, elastin, alginate, and combinations thereof disposed on the flexible membrane;
   (b) applying one or more pressurized fluids to the pneumatic chamber using the pneumatic actuator, thereby altering the shape of the flexible membrane through mechanical stimulation and modulating bending stress on the cells; and
   (c) analyzing the cells in the cell culture apparatus.
Clause 31. The method of clause 30, wherein the one or more cell types are cultured on the hydrogel, within the hydrogel, or a combination thereof.
Clause 32. The method of clause 30 or 31, wherein two or more different cell types are co-cultured within the hydrogel.
Clause 33. The method of any one of clauses 30-32, wherein the cell culture apparatus further comprises one or more perfusion channels, perfusion channel inlets, and perfusion channel outlets each fluidly connected to the hydrogel and configured to deliver one or more liquid fluids to the hydrogel.
Clause 34. A cell culture apparatus comprising:
   (a) one or more cell culture chambers each comprising a flexible membrane comprised of one or more microfluidic layers each independently comprising one or more microfluidic chambers, microfluidic inlets, and microfluidic outlets disposed therein, each of the microfluidic chambers being fluidly connected through one or more porous membranes;
   (b) an extracellular matrix layer disposed within each of the one or more cell culture chambers, the extracellular matrix layer comprising a hydrogel selected from the group consisting of collagen, elastin, alginate, and combinations thereof;
   (c) a means for bonding or injecting the hydrogel to the flexible membrane to form a three-dimensional (3D) cell culture environment;
   (d) one or more perfusion channels, perfusion channel inlets, and perfusion channel outlets formed around the hydrogel and configured to deliver one or more cell culture media to the hydrogel;
   (e) a means for removing cell culture media from at least one cell culture chamber to enable air exposure, thereby initiating cell differentiation; and
   (f) a means for exposing the hydrogel to various biomechanical stimuli.

### EXAMPLES

### Example 1

### Functional Cornea Organ-Chip Model

### Materials and Methods

### Human Endothelial Cell Culture

Immortalized endothelial cells (HCEC-B4G12, ACC 647, DSMZ) were cultured. In T25 culture flasks coated with a 1:1 mix coating solution containing 100 μg/mL collagen type I and 1 mg/mL fibronectin solution derived from human, the HCEC cell population were grown in the serum-free medium of Human Endothelial-SFM (Gibco Invitrogen) supplemented with 10 ng/mL human recombinant bFGF. Serum-free cultured cells were passaged using trypsin/EDTA (0.05%/0.02%). The enzyme activity was inhibited by adding a 500-fold dilution of a proteinase inhibitor cocktail (Sigma). Cells were seeded at a density of 2×10⁵ cells/cm² on the artificial cornea template and cultured at 5% CO₂/37° C. The medium was changed three times per week.

### Human Keratocyte Cell Culture

Primary keratocytes (HCK, 6520, ScienCell) were cultured in serum-starved medium containing DMEM:F12 (Life Technologies) supplemented with 1% penicillin/streptomycin, 1% L-ascorbic acid 2-phosphate (Sigma-Aldrich), and 1% Insulin-Transferrin-Selenium solution. Trypsin-EDTA (0.025%) (Cat. #0183, ScienCell) was used to harvest serum-starved keratocytes, which were then neutralized using TNS solution (Cat. #0113, ScienCell). A 1×10⁶ cells/mL cell suspension was loaded into a human collagen type I matrix.

### Human Epithelial Cell Culture

Immortalized human corneal epithelial cells (HCE-2, CRL-11135, ATCC) were cultured in serum-free keratinocyte medium (KSFM) with 5 ng/mL human recombinant EGF, 5 ng/mL insulin derived from human, 4 μg/mL XerumFree™ supplement (XF212, TNCBIO), and 500 ng/mL hydrocortisone. Serum-free cultured cells were passaged using 0.05% (w/v) Trypsin-EDTA (0.53 mM). A 3×10⁶ cells/mL cell suspension was seeded on top of the self-assembled stroma using a droplet seeding system of BIO X, CELLINK with the following conditions: pressure=5 kPa; needle size=25 G; extrusion time=0.03 s; and speed=100 mm/s. All media were changed once every 2 days and were cultured at 5% CO₂/37° C.

### Fabrication of the Artificial Cornea Template

The artificial cornea template has the function of transforming the planar surface into a cornea-like curved shape. The template is composed of a fluidic pneumatic chamber made of silicone polymer PDMS, a thin PDMS membrane layer for cell culture, and a medium reservoir to supply medium to the cells. The thin PDMS layer is flexible, allowing it to be inflated and take on a convex curvature by the fluidic pneumatic chamber as liquid is infused continuously through the chamber and the residual air is expelled from the original fluidic chamber. The templates were sterilized with 80% ethanol and deionized (DI) water before pretreatment.

### Layer-by-Layer Assembled PDMS Surface

To modify and enhance the PDMS surface of the artificial cornea template for covalent binding with collagen and fibronectin mix solution from human origins, dopamine hydrochloride (Sigma-Aldrich) was modified by 5 mg/mL concentrations in 0.01% (w/v in 1M Tris-HCL buffer, pH 8.5) dopamine solution (Sigma-Aldrich) for 24 hours. The artificial cornea template was then washed twice with DI water and was further immersed in a 1:1 mix solution containing 100 μg/mL (in DI water) collagen type I (Advanced BioMatrix) and fibronectin (human plasma, Sigma-Aldrich) overnight at 37° C. for collagen coating. Following this, the coating solution was removed, and the surfaces of the template were washed twice with DI water. The templates were sterilized with UV before cells were cultured.

### Cell Viability Assays

Cell viability was assessed by the Cell Counting Kit-8 (CCK-8, Sigma) according to the manufacturer's protocol. Immortalized endothelial cells were seeded on the artificial cornea template coated with dopamine (1, 3, 5 mg/mL) for serving a suitable environment at the density of 2×10⁴ cells/cm². CCK-8 solution (10 μL) was added to each sample, and cells were incubated at 37° C. for 2 hours. The absorbance at 450 nm was measured using a multimode reader.

### Three-Dimensional (3D) Corneal Stromal Constructs

Stromal constructs generated by human corneal keratocytes ere cultured in 3D collagen matrix. To obtain a final collagen concentration of 0.8 mg/mL at pH 8.0, solutions of acid-solubilized human type I collagen (Advanced BioMatrix, Vitrogel 3 mg/mL) were tuned using 10×PBS and sterile 0.1 M NaOH to neutralize the pH of the resultant liquid. The constructs were then incubated at a temperature of 37° C. for 90-120 minutes to form a gel.

### Air-Lifted Exposure Culture

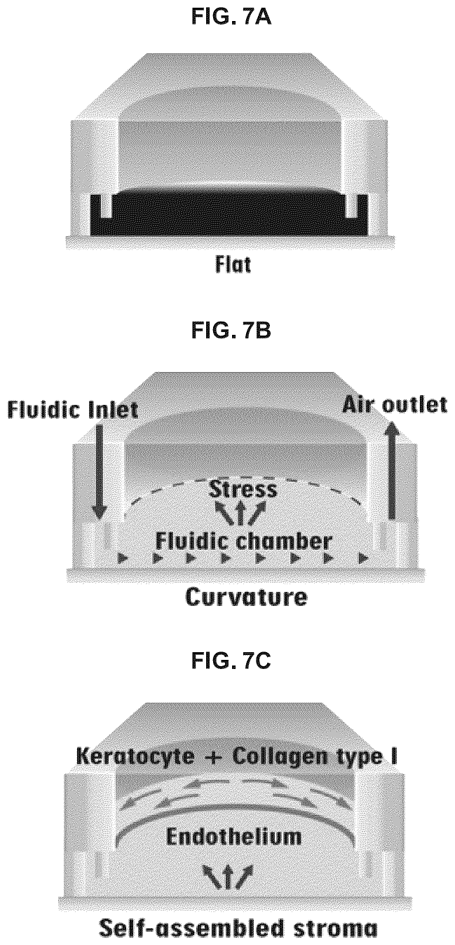

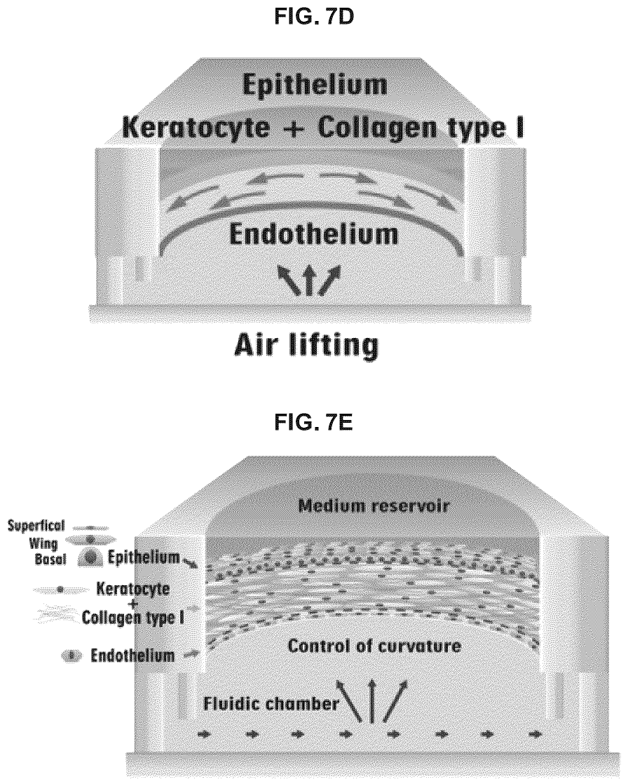

For the air-liquid interface, immortalized human corneal epithelial cells were cultured on top of the self-assembled stroma by filling the medium reservoir with KSFM medium containing high calcium (1.15 mM CaCl₂) until it fully covered the top of the collagen matrix. After the cells were grown to be fully confluent, the medium was removed from the apical layer of the template, exposing the cells to an air-liquid interface (FIG. 7D). The medium was changed every day during air lifting for 7 days.

### Monitoring Cell Phenotypes and Behaviors on the Curvature

The cells in each layer were analyzed via immunostaining. The samples were washed with PBS briefly, fixed in 4% paraformaldehyde for 15 min, and then washed three times with PBS. After incubating with blocking buffer (12411, Cell Signaling Technology) at room temperature for 1 hour, the samples were incubated with primary and secondary recombinant antibodies. The primary antibodies were recombinant anti-RhoA (ab187027, Abcam), ARPC2 (ab133315, Abcam), Arp3 (ab181164, Abcam), vinculin (ab129002, Abcam), and α-SMA (ab124964, Abcam). The secondary antibodies were recombinant superclone Alexa Fluor 488 (A27034, Thermofisher), Alexa Fluor 647 (A28181, Thermofisher), and Alexa Fluor 555 (A27017, Thermofisher).

### Co-Culturing Cells on the Artificial Cornea Template

Immortalized human endothelial cells were seeded on the planal artificial cornea template coated with the described layer-by-layer method using dopamine, fibronectin, and collagen. Fully confluent endothelial cells were monitored by Cell Tracker™ Green CMFDA (C7025, Invitrogen™) and phase contrast imaging to confirm single layered corneal endothelium. The monolayered hexagonal structure of endothelium could be identified through F-actin staining and phase contrast. The mixture of collagen type I and corneal keratocytes were loaded on the endothelial cell covered template and incubated at 37° C. for gelation. The natural extracellular matrix (ECM) generated by endothelial cells during full confluency enables the collagen matrix to adhere closely to the endothelium. By injecting pressurized fluid into the pneumatic chamber, the attached collagen matrix and endothelium became curved.

The organized cell distribution and expression of self-assembled collagen by curvature was assessed by F-actin, Hoechst, and collagen type I staining. Further, the co-culture conditions for the 3D self-assembled collagen matrix laden corneal keratocytes were optimized. Co-culture medium was used in a 1:1:1 mixture for endothelial, keratocytes, and epithelial cells. Cell viability was confirmed for cells cultured in three chambers of a microfluidic chip by staining with Cell Tracker™ Green CMFDA (C7025, Invitrogen™) and Deep Red Dye (C34565, Invitrogen™) for live cell monitoring. A CCK viability assay may also be applied to further characterize the co-culture systems.

Epithelial cells were seeded on the top of the template contained self-assembled collagen and endothelium using droplet seeding, where a stable cell number (±3.96% standard deviation), seeding area (±3.9% standard deviation), and cell density (±6.65% standard deviation) were achieved. Epithelial cells were cultured with KSFM medium containing high calcium to induce cell differentiation while generating the air lifting interface. The epithelial cells were submerged 80-90% in medium using the advantage of curvature shape. The superficial, wing, and basal cell layers could be identified through an epithelium marker. In addition, hematoxylin and eosin (H&E) staining could be performed to compare with a real human cornea to show the similarity of the morphology between the reconstructed cornea tissue and the cornea in vivo using techniques such as scanning electron microscopy.

### Validation of Membrane Formation

Before inducing an inflammatory infection for validation of membrane genesis, the functionalized layer is confirmed via transepithelial/transendothelial electrical resistance (TEER) measurement. An epithelial Volt/Ohm meter device (EVOM2, WPI) for TEER is used to measure the value of TEER on apical and basal sides of the artificial cornea.

Monitoring Membrane Genesis with ECM Deposition

The cells in each layer were analyzed via immunostaining. The samples were washed with PBS briefly, fixed in 4% paraformaldehyde for 15 min and then washed three times with PBS. After incubating with blocking buffer (12411, Cell Signaling Technology) at room temperature for 1 hour, the samples were incubated with primary and secondary antibodies. Primary antibodies: recombinant anti-laminin beta (ab273053, abcam) and TSP-1 (ab267397, abcam); and secondary antibodies: recombinant superclone Alexa Fluor 488 (A27034, Thermofisher) and Alexa Fluor 555 (A27039, Thermofisher).

### Lipopolysaccharide (LPS) Exposure

To confirm the formation of a membrane between each layer and the function of the corneal layer, the artificial cornea is damaged with LPS four weeks after culturing in co-culture. The epithelium of the artificial cornea is treated with LPS at 1 μg/mL, 5 μg/mL, and 10 μg/mL and membrane formation is investigated by observing stroma and endothelium.

### Quantitative Analysis of the Developed Artificial Cornea

To verify the function of the generated membrane, the LPS treated artificial cornea is evaluated for cytotoxicity (e.g., CCK-8 assay), cell viability (e.g., Live/Dead cell assay), histological staining (e.g., H&E staining, Periodic acid-Schiff (PAS) staining, and Masson's Trichrome staining), and TEER to quantify the damaged cornea with membrane. The organization of proteoglycans in the membrane is also determined using confocal microscopy to analyze the collagen level (Col-F, Immunochemistry). qRT-PCR analysis was also used to assess the expression levels of genes associated with junctional proteins, proteoglycans, stromal keratocytes, and myofibroblasts. In addition, the activated stroma by epithelium infection is analyzed by immunostaining using recombinant antibodies for studying wound healing mechanisms in stroma related to the membrane's function.

### Example 2

Promoting the Integrity of an Artificial Cornea with Curvature

Studies were conducted to develop a controllable cornea curvature template and interfacial membrane formation that creates a self-assembled collagen matrix under airlifting environment required for constructing a functional artificial cornea (FIG. 7A-E). Previous studies successfully designed and developed a cornea organ-chip that can reproduce the morphological and topographical properties of the corneal epithelium, basement membrane, and Bowman's layer. This model allows drug mass transport investigation in three different flow conditions (static, continuous flow, and pulsatile flow) using two ocular drugs. With this work, mechanical factors were applied to recapitulate stroma. Specifically, strain and stress were applied through the curvature to a collagen matrix loaded with keratocytes to induce the collagen fiber and cell orientation into polarized similar with natural human cornea (FIG. 7B). The epithelium and endothelium reconstructed using primary epithelial and endothelial cells was incorporated on to the top and bottom of the collagen matrix (FIG. 7E) so that fully stacked cornea tissue interacted with each other and generated a cell-to-cell membrane. This approach overcomes the limitations of the artificial cornea caused by synthetic materials on biocompatibility and biodegradability. The tissue-engineered cornea is able to recapitulate these properties to maintain the function of cornea. Specifically, epithelium had three different phenotypes of epithelial cells with different density and shapes (basal, wing, and superficial cells) by applying air interface on epithelium, stroma had an orthogonal collagen lamellae structure with polarized cell, and endothelium maintained the hydration level via the ‘pump leak’ mechanism. This model allows for the investigation of numerous corneal diseases prior to clinical surgery by using an artificial cornea in replacement of an animal cornea, as well as the prospect of evaluating the side effects and toxicity of various medications.

One main purpose of this study was to explore how to reconstruct a functionalized artificial cornea by understanding cellular responses on curvature (i.e., curvotaxis) and membrane formation from cell-cell interaction of each layer to resolve the current issues of keratoprosthesis (KPro). Based on previous co-culture studies on transwells, two hypotheses were formulated: (1) the curvature plays a key role for ECM alignment and cell alignment; and (2) the interaction of epithelium and stroma, as well as stroma and endothelium, enables the formation of a Bowman's layer and Descemet's membrane to promote integrity of cornea.

### Self-Assembled Stroma by Curvature

Polydimethylsiloxane (PDMS) surface was modified for binding with collagen type I and human plasma fibronectin to culture endothelial cells, and the function of a single layered endothelium cultured on a curved artificial cornea template was validated as compared to a flat template surface.

By applying curvature to an artificial cornea template (collagen gel), the self-assembled stroma was constructed using a mixture of collagen type I hydrogel and corneal keratocytes (FIG. 7C). Curvature affects cell migration, cell polarization, and proteoglycan rearrangement as a result of focal adhesion and activation of the Rho signaling pathway. To confirm self-assembled stroma, second harmonic generation microscopy (SHG) and scanning electron microscopy (SEM) were used to examine collagen and proteoglycan rearrangement. The gradient in cell density between the anterior and posterior stroma of the cornea was also evaluated using sectioning samples for histological staining.

### Reconstruction of Epithelium

The effects of curvature on distributing collagen fibers into alignment were demonstrated, and corneal stroma similar to a real cornea were emulated, which has a gradient of cell density depending on the specific location of cornea.

On the self-assembled collagen hydrogel, epithelial cells were seeded and subsequently applied to an air-lifting condition. The collagen matrix can be maintained with moisture from the medium around it, enabling epithelial cells growing on top of it to be cultured in an airlifting environment (FIG. 7D). The air-lifted epithelium was comprised of different cell shape and density; basal cells, wing cells, and superficial cells. The functionality of epithelium can be confirmed by measuring TEER. To further understand the curvature effects, the cell behaviors in all layers were examined utilizing immunostaining with recombinant antibodies (e.g., RhoA, ARPC2, Arp3, vinculin, and α-SMA).

### Functional Endothelium

Co-culture systems on the self-assembled collagen matrix were established, and an airlifting environment was produced and validated to reconstruct the epithelium, which has three types of epithelial cells.

On the convex side of the self-assembled collagen hydrogel, endothelial cells were seeded to form a single-layered endothelium. The endothelium comprised of hexagonal cells is critical in determining endothelial function. By observing hexagonal shape, cell size homogeneity, and junction shape, the phenotype of corneal endothelial cells on the curved hydrogel is able to be confirmed through resources like MATLAB, as well as cell viability. In addition, a TEER measurement can be conducted to assess the function of the monolayered endothelium affected by tight junction formation, and ion transportation from ATPase pumps.

### Example 3

Interfacial Membrane Formation from Cell-Cell Interaction

The functionalized artificial cornea was investigated, including three different cell types of the epithelium, organized stroma, and monolayered hexagonal shape of the endothelium. To confirm the completely constructed artificial cornea, the TEER values of functionalized and non-functionalized artificial corneas are compared. Epithelium are targeted with a suitable concentration of LPS, and the stromal response to epithelial infection is investigated to verify the creation of a membrane.

Airlifted epithelium is considered to be the significant source of epithelial basement membrane formation since the differentiated epithelium secretes extracellular vesicles (EVs). EVs communicate between cells by transporting proteins, metabolites, and nucleic acids. The EVs from cornea epithelium stimulate stromal fibrosis making the keratocytes differentiate into myofibroblast, and also secrete ECM proteins such as fibronectin, collagen, and laminin, as well as thrombospondin-1 (TSP-1), which have roles in anchoring ECM proteins. These ECM depositions play essential roles in creating the interfacial membranes. These secretions and depositions follow similar patterns in co-cultures of endothelial and stromal cells so that these cell-cell communications enable the formation of a membrane between each cell layer.

To validate the membrane formation between cornea cell layers, the ECM components and stromal fibrosis generated by airlifted epithelium and the cell-cell communication were investigated through immunostaining with recombinant antibodies (e.g., laminin beta 1, and TSP-1). To observe any structural alteration between cell layers, TEER measurement is used to compare the non-functionalized artificial cornea to understand the epithelium's barrier function, membrane, and endothelium.

An inflammatory infected disease model is created by adding LPS targeted to epithelium for validation of the membrane formation. The effect of a membrane as a physical barrier from inflammatory infection is evaluated to compare with membrane-free artificial cornea. The differences between membrane-free and membrane-formed artificial corneas is revealed by the response of keratocytes in the stroma and endothelium from LPS treatment. A CCK-8 assay or a Live/Dead cell staining assay is conducted to confirm a suitable concentration of LPS for targeting the epithelium and to investigate the reaction of endothelium from infection. To evaluate activated keratocytes and rearrangement of extracellular matrix in stroma generated by infected epithelium, real-time PCR was conducted using markers related to RhoA activity and proteoglycans, as well as the transmittance of light using UV-Vis spectroscopy.

### Example 4

### Results and Advantages of the Functional Cornea Organ-Chip Model

A pneumatically controlled organ-chip was developed to mimic corneal diseases with varied curvatures to study the effects of curvature on the cornea wound healing process and stroma regeneration. Results showed that cell migration, collagen secretion, and cell polarization were promoted with increasing curvature, activating the Rho and Rac pathways.

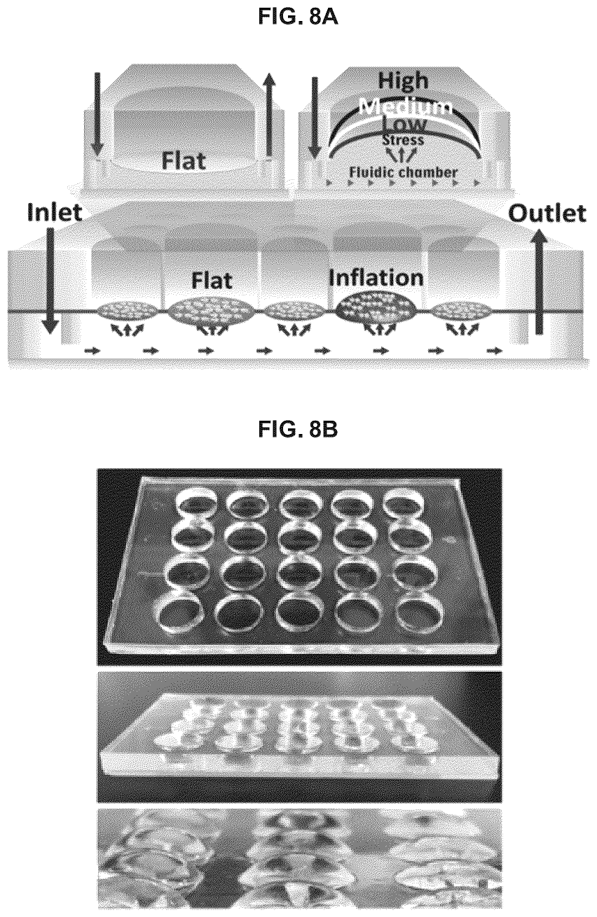

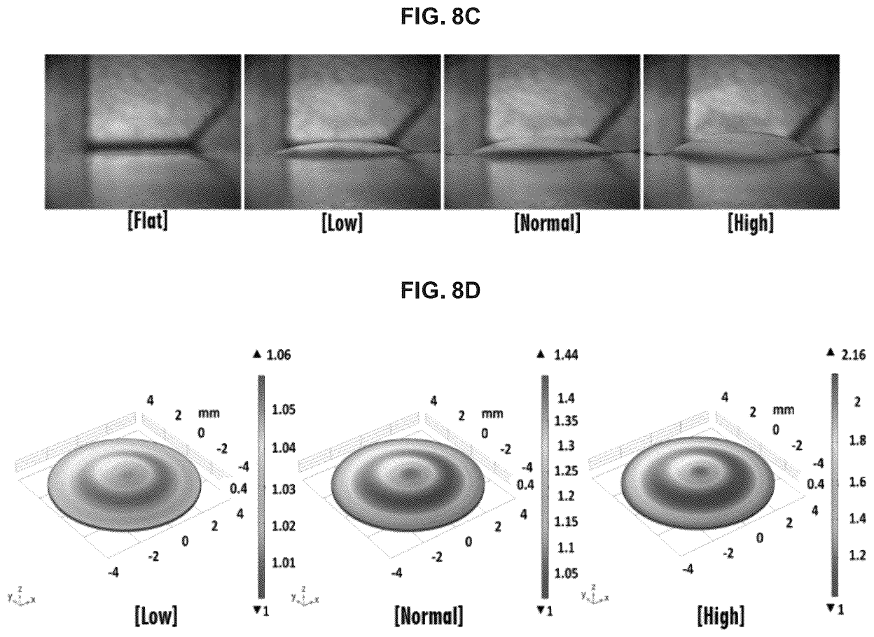

FIG. 8A shows a schematic illustration of a pneumatical array balloon chip as described herein for a cornea organ-chip model. FIG. 8B shows an example array chip having multiple cell culture chambers on a single plate system. FIG. 8C shows side view images of different membrane curvatures comparing flat, low, medium (i.e., “normal”), and high following mechanical stimulation through a pneumatic chamber. FIG. 8D shows finite element analysis (FEA) simulation plots estimating strain profiles for the different membrane curvatures shown in FIG. 8C.

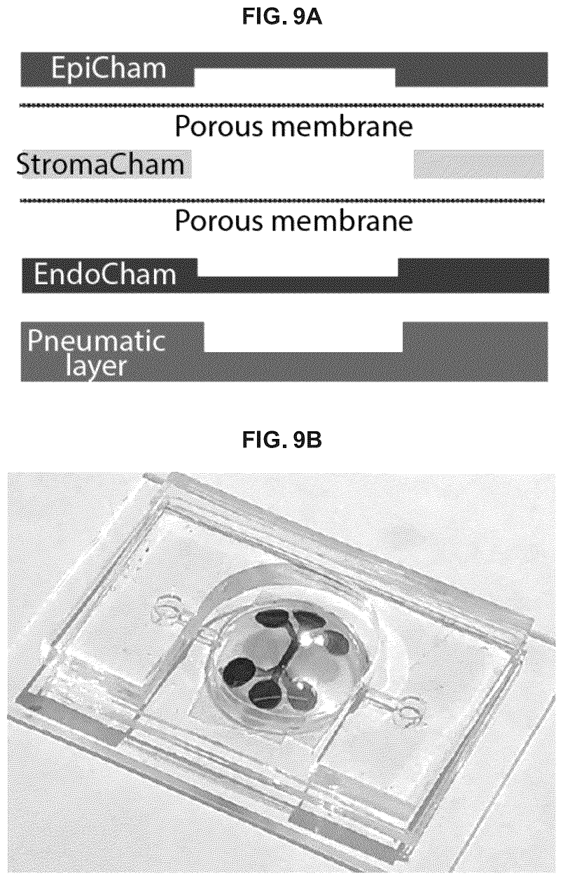

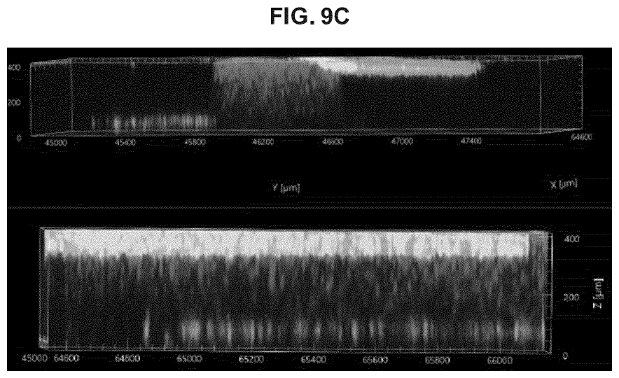

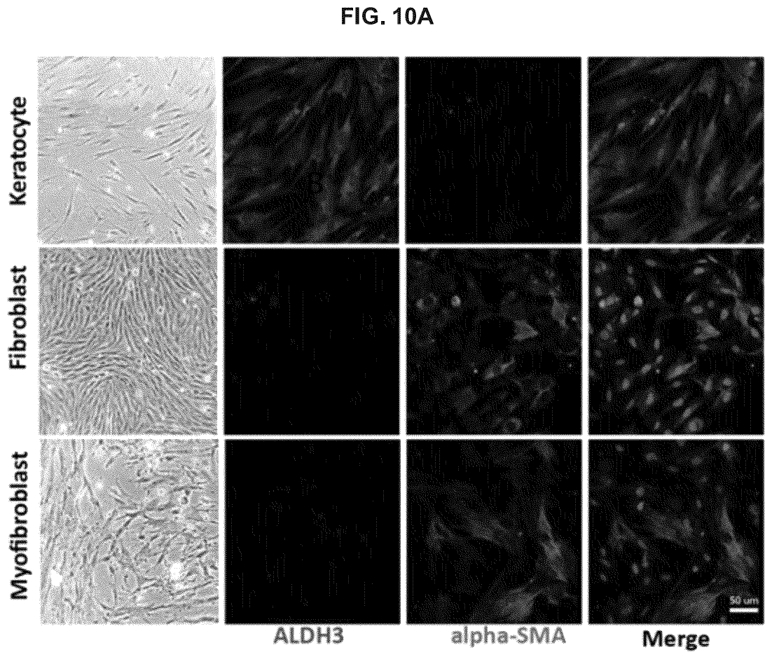

FIG. 9A-C show an example of a cell culture apparatus comprising a flexible membrane having multiple microfluidic layers and chambers for culturing different cell types of a cornea. In FIG. 9A, the red microfluidic layer comprises an epithelial cell chamber, the yellow microfluidic layer comprises a stromal cell chamber, and the blue microfluidic layer comprises an endothelial cell chamber, all separated and fluidly connected by porous membranes. Below the flexible membrane is a pneumatic layer comprising a pneumatic chamber for membrane stimulation. FIG. 9B shows an example cell culture apparatus organ-chip comprising a flexible membrane having the three different microfluidic layers and cell types as depicted in FIG. 9A. FIG. 9C shows a representative confocal microscopy image of a three-layered chip with immortalized human epithelial cells stained with CellTracker™ Green CMFDA Dye (top and bottom cell layers) and immortalized human keratocytes loaded with 10% GELMA hydrogel stained with CellTracker™ Deep Red Dye (middle cell layer).

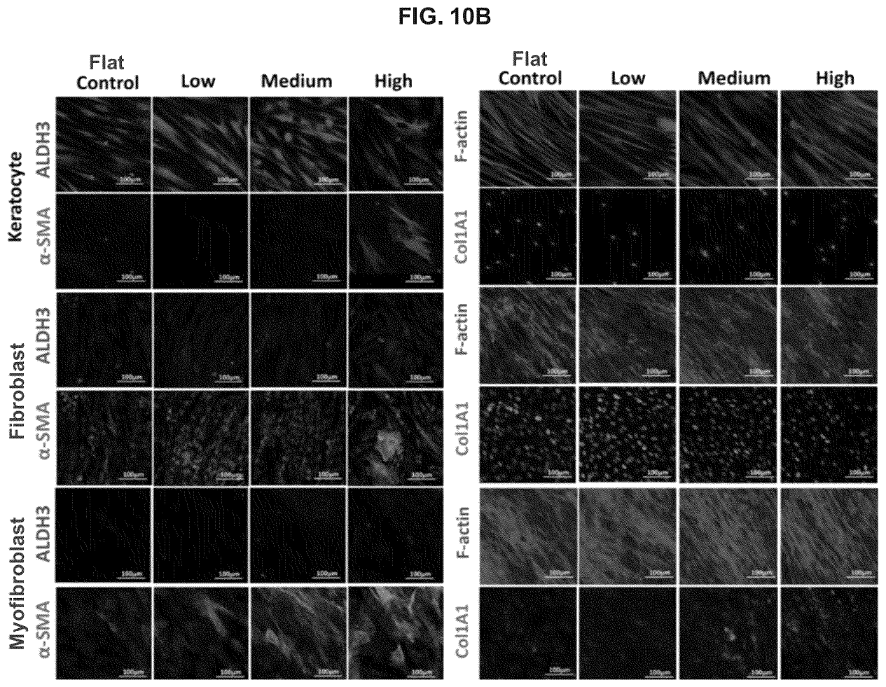

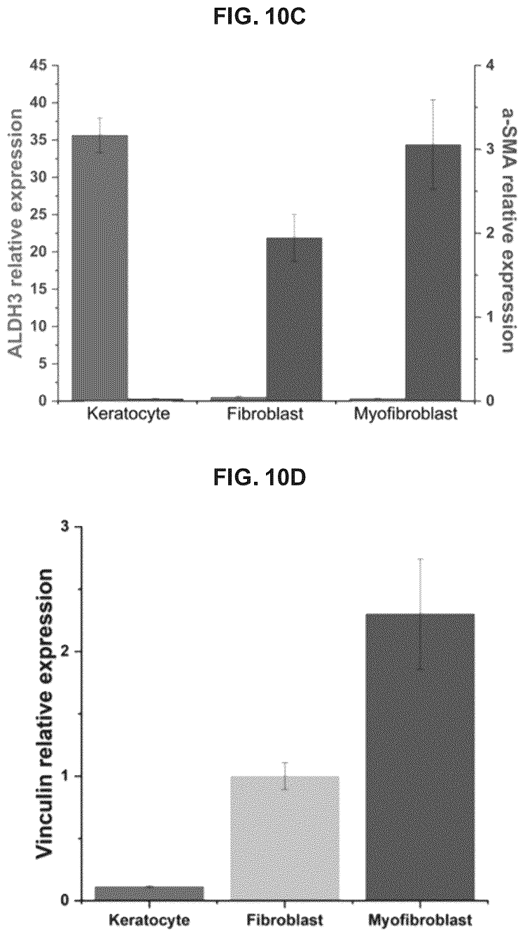

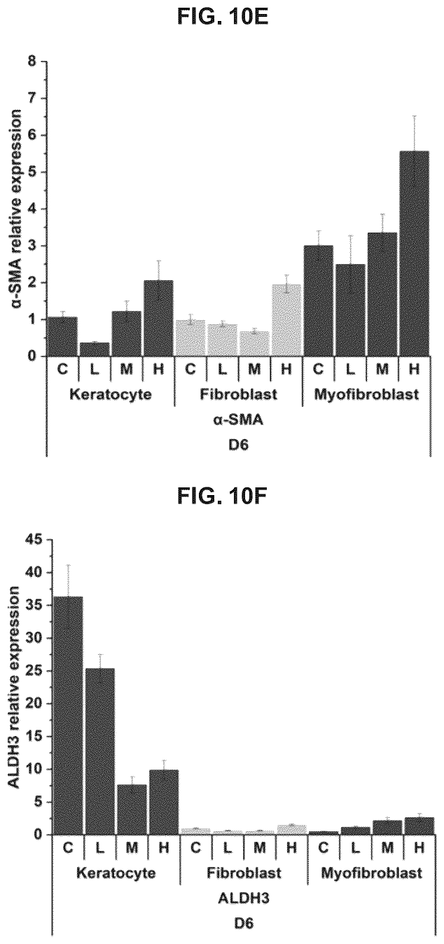

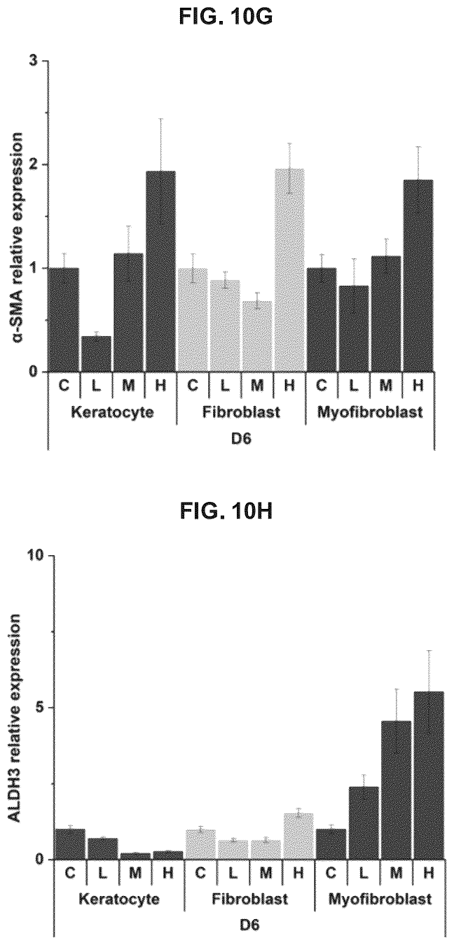

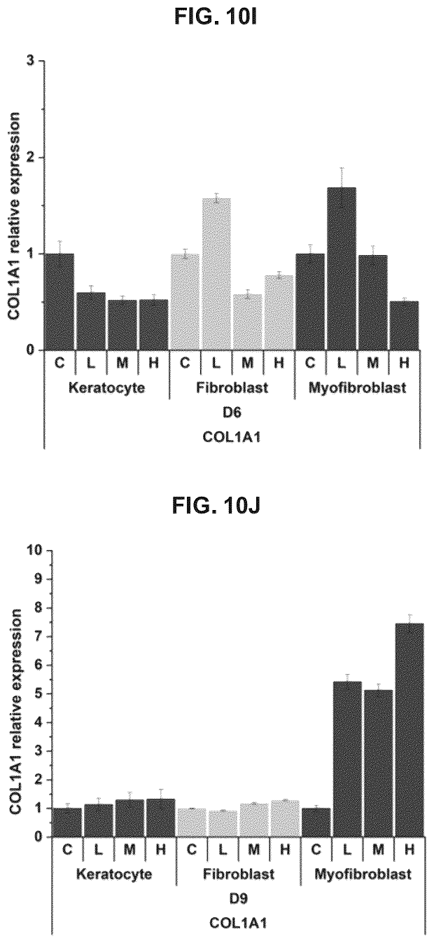

FIG. 10A shows representative immunofluorescence images of keratocytes, fibroblasts, and myofibroblasts stained for ALDH3 and α-SMA phenotype markers. Scale bars: 50 μm. FIG. 10B shows representative immunofluorescence images of keratocytes, fibroblasts, and myofibroblasts stained for ALDH3, α-SMA, collagen type I, and F-actin phenotype markers under different curvatures of flat control, low, medium, and high using the cell culture apparatuses as described herein. Scale bars: 100 μm. FIG. 10C-D show immunofluorescence quantitative analysis of the relative expression of ALDH3, α-SMA, and vinculin (focal adhesion marker) in keratocytes, fibroblasts, and myofibroblasts prior to applying any membrane curvature through pneumatic actuation. FIG. 10E-J show immunofluorescence quantitative analysis of the relative expression of ALDH3, α-SMA, and collagen type I phenotype markers in keratocytes, fibroblasts, and myofibroblasts with flat control (C), low (L), medium (M), and high (H) membrane curvatures.

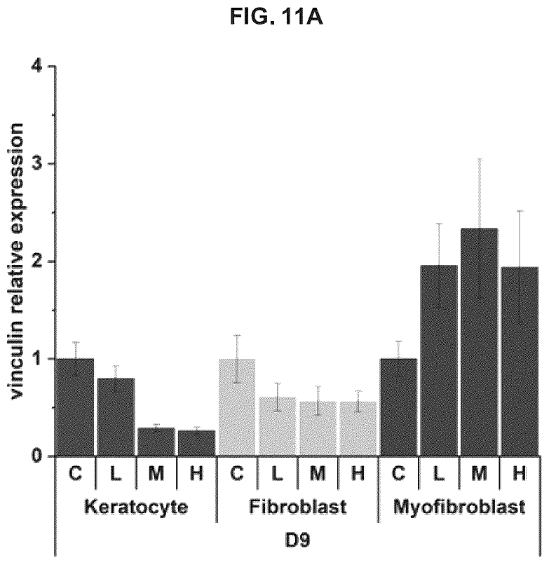

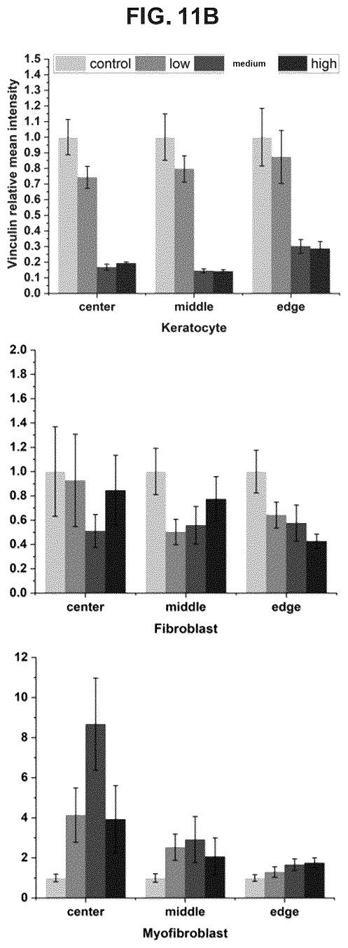

FIG. 11A shows immunofluorescence quantitative analysis of the relative expression of vinculin (focal adhesion marker) in keratocytes, fibroblasts, and myofibroblasts with flat control (C), low (L), medium (M), and high (H) membrane curvatures. FIG. 11B shows immunofluorescence quantitative analysis of the relative mean intensity of vinculin (focal adhesion marker) at specific cell loci (center, middle, or edge) in keratocytes, fibroblasts, and myofibroblasts with flat control, low, medium, and high membrane curvatures.

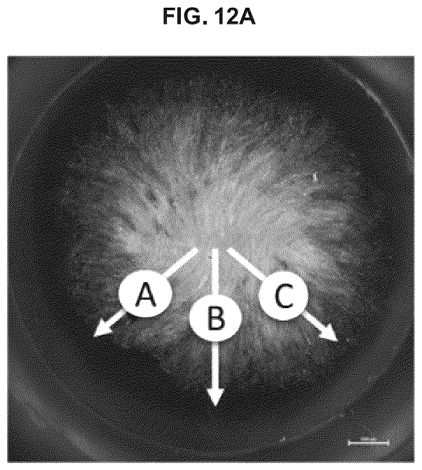

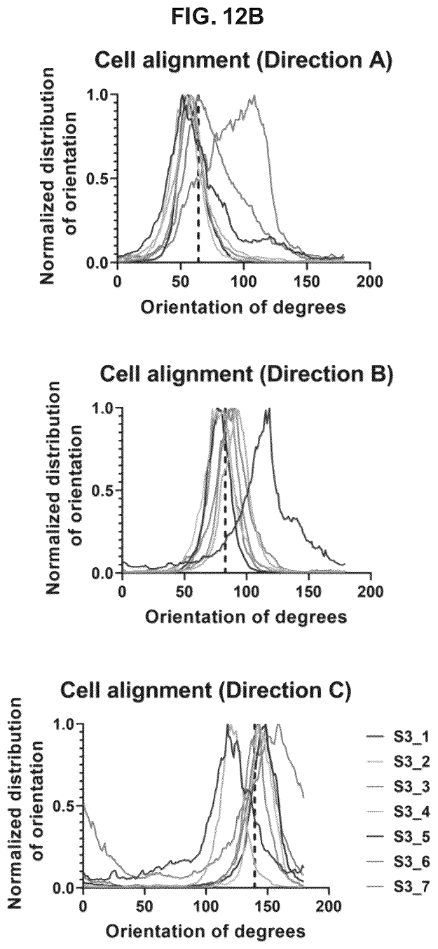

FIG. 12A-B show quantitative analysis of cell alignment/orientation depending on the specific direction (A, B, or C) using ImageJ processing. FIG. 12B shows graphs of the cell orientation by the location of curvature for each direction. S3_1=basal, lower; and S3_7=apical, higher.

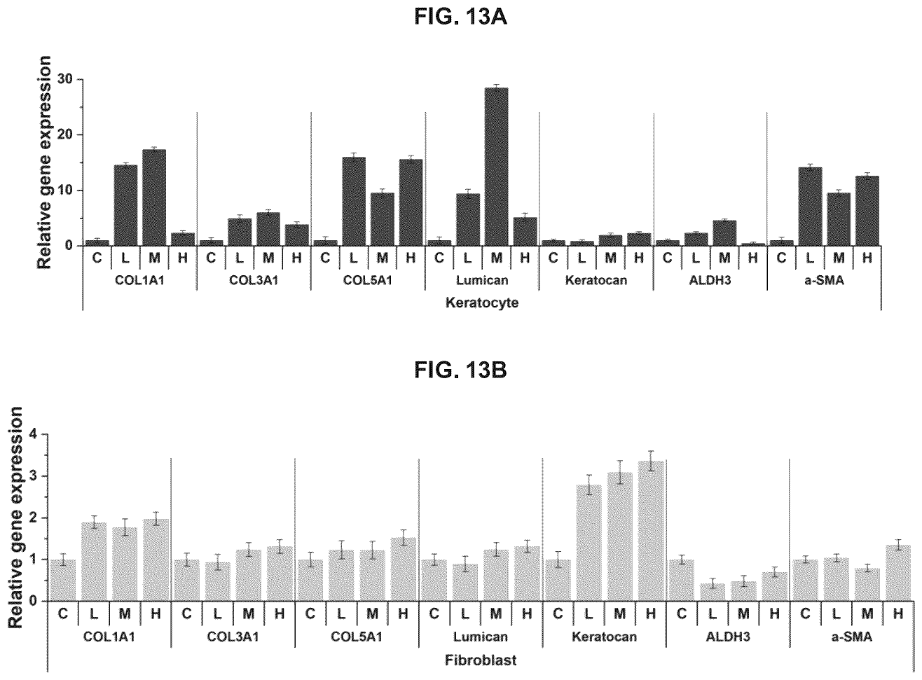

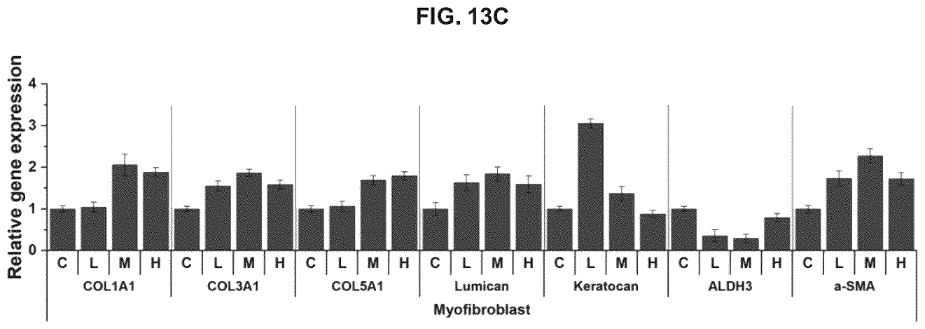

FIG. 13A-C show immunofluorescence quantitative analysis of the relative expression of various phenotype markers in keratocytes (FIG. 13A), fibroblasts (FIG. 13B), and myofibroblasts (FIG. 13C) with flat control (C), low (L), medium (M), and high (H) membrane curvatures.

The use of a pneumatically controlled cell culture apparatus chip for studying the impact of corneal curvature on wound healing offers several advantages. The cornea chip allows for the precise control of curvature, which is crucial in mimicking the mechanical forces and extracellular matrix (ECM) arrangement of the cornea. This pneumatic control configuration can be used to investigate the effects of different curvatures on cell behaviors which affect cell migration, polarization, and activation processes. In particular, the pneumatically controlled cornea chip offers a significant advantage over traditional methods in that it can emulate the three-dimensional (3D) structure of the cornea by adjusting the height of the z-axis. This means that the chip is not only able to reproduce the curvature of the cornea, but it also mimics the shape and depth of the cornea, which is critical for studying the impact of corneal curvature on wound healing. In comparison, traditional methods for studying corneal curvature merely involve the reconstruction of two-dimensional (2D) grooves or ridges on flat surfaces, which limits the ability to fully replicate the 3D structure and depth of the cornea. By using the pneumatically controlled cell culture apparatus chip to emulate the 3D structure of the cornea, it is possible to gain a more complete understanding of the impact of curvature on cell behavior and molecular mechanisms involved in wound healing. This research may lead to the development of new and more effective treatments for corneal diseases such as keratoconus, plana, and keratoglobus.

The disclosed pneumatically controlled cell culture apparatus cornea organ-chip provides a platform for studying cell behavior under conditions that closely resemble the in vivo environment of the cornea. This is because the chip can create a 3D environment that allows cells to interact with the ECM in a manner that more closely mimics the in vivo environment of the cornea. The ECM is an important component of the cornea that provides mechanical support and regulates cell behavior. In addition, the curvature of the cornea affects the mechanical factors of stress and strain, which play a critical role in regulating overall corneal cell behaviors. In particular, these factors can affect cell orientation and cell organization in the corneal stroma. By studying cell behavior under different curvature conditions, it is possible to gain insight into the impact of stress and strain mechanical factors on corneal wound healing. For example, the disclosed pneumatically controlled chip is able to be used in studying the effects of curvature on cell morphology, focal adhesion protein expression, and cytoskeleton and nuclei polarization. Researchers have observed that higher curvatures induce extended and spindle-shaped cell morphologies, regardless of the cell phenotype. Additionally, vinculin expression, which is a focal adhesion protein, increases with higher curvatures. Furthermore, for most curved cases, the cytoskeleton and nuclei polarize in the direction of the curvature, even with a low strain profile. This knowledge of the effects of stress and strain mechanical factors on cell behavior may lead to the development of new and more effective treatments for corneal diseases such as keratoconus, plana, and keratoglobus.

The disclosed pneumatically controlled cell culture apparatus cornea organ-chip allows for the investigation of the molecular mechanisms involved in wound healing under different curvature conditions. This is because the organ-chip can be used to study gene expression using qRT-PCR with phenotype-specific markers and ECM specific markers. The ability to investigate the molecular mechanisms involved in wound healing under different curvature conditions may lead to the development of new treatments for corneal diseases. In addition to studying gene expression using qRT-PCR, the pneumatical control chip also allows for the investigation of protein expression and localization using immunostaining techniques. By analyzing protein expression and localization in response to different curvatures, researchers can gain insight into the impact of corneal curvature on wound healing at the protein level. By performing immunostaining on cells cultured on the pneumatically controlled chip, researchers can investigate the effects of curvature on protein expression and localization in a controlled and reproducible manner.

The disclosed pneumatically controlled cell culture apparatus cornea organ-chip also offers the advantage of being able to study the effects of different curvatures on cell behavior over a range of physiological conditions, such as changes in intraocular pressure or during the natural growth and development of the cornea. Additionally, the chip can be controlled to create different curvature profiles, mimicking the natural variations in corneal curvature that occur due to cornea shape deformation. By studying the effects of dynamic changes in corneal curvature on cell behavior, researchers can gain a more comprehensive understanding of the wound healing process and the factors that influence it. This knowledge may lead to the development of new and more effective treatments for corneal diseases.

Using a pneumatically controlled chip for studying the impact of corneal curvature on wound healing provides a precise and reproducible platform for investigating the effects of curvature on cell behavior and molecular mechanisms. An increase in cell phenotype changes and in ECM secretion due to mechanical factors induced by curvature was demonstrated. It was also confirmed that the phenotype of quiescent keratocyte transformed into myofibroblastic characteristics and increased collagen secretion. These results suggest that curvature can modulate the cell phenotype and cell behaviors. Also, the cultured cells on the high curvature showed organized cell morphology compared with the low and flat curvature. This means that curvature affects cell organization, which is a crucial fact in the stroma due to the stroma having an organized collagen lamellae structure. These properties have a significant relation with the transparency of the cornea. Therefore, the fabrication and curvature characterization process allow for the investigation of the impact of corneal curvature on wound healing under different physiological conditions, as well as the investigation of protein and gene expression using qRT-PCR and immunostaining techniques. The resulting data may lead to new insights into regulating corneal wound healing and developing new treatments for corneal diseases such as keratoconus, plana, and keratoglobus. The pneumatically controlled cornea organ-chip provides a promising avenue for advancing our understanding of corneal biology and improving treatment outcomes for corneal diseases.

### Example 5

### Functional Heart Valve Organ-Chip Model

Heart valve structure and dynamics must be thoroughly evaluated at the molecular level to understand the pathophysiology of valvular diseases. A heart valve exhibits a dynamic motion that is passively generated by various pulsatile flow profiles to transport blood without significant loss of volume. Forces are exerted on heart valves as a compound effect of tension, compression, shear, and flexure. Flexural bending and shear stresses play a major role in valvular tissue and cell physiology, and valvular endothelial cells (VECs) and valvular interstitial cells (VICs) are known to respond to these stimuli, mainly by aligning extracellular matrices (ECM) to maintain structural integrity. Strict mechanobiology studies of valvular tissue have evaluated responses of native and prosthetic aortic valves to blood flow in vivo and by using in vitro models. However, studies correlating mechanical stimuli and molecular responses issued by valvular cells are less comprehensive. In addition, it is widely recognized that conditions of high mechanical stress such as endothelium denudation trigger pathological change.

Results of flexural bending and shear studies have broadly reported that the valvular endothelium protects the valve leaflets from flow-induced damage, either by sending signals to VICs to trigger molecular compensatory mechanisms or by simply providing a physical barrier against stresses. However, the exact mechanism by which VECs protect the leaflet remains unknown. Flexural bending stimuli also play critical roles on overall mechanobiology of valvular cells. However, not much is known about biological responses of valvular cells to flexural bending stress, perhaps due to the difficulty of generating a reasonable in vitro model that is able to properly mimic valvular flexure. Specifically, studies have shown that flexure induces ECM alignment; therefore, VECs may follow that alignment to form mechanically stable endothelium. Under VECs, type 1 collagen is aligned circumferentially in response to valvular flexure during the cardiac cycle. VECs also align in the same direction as the collagen. VEC alignment is impacted by ECM directional cues and shear stress.

Previous investigations have also been performed to study how shear and flexure are coupled. Specifically, alignment phenomena were investigated using an integrated microfluidic platform, including multiple microvalves, to generate a wide range of pulsatile flow patterns. These studies confirmed that VECs align perpendicular to the flow without any aligned ECM. These results elicited questions on how VECs adhere to collagen fibers, especially during valvular calcification. In calcific aortic valve disease (CAVD), VECs are believed to be recruited into the ECM, acquire an activated phenotype, and remodel the damaged inner tissue and continuously deposit collagen, resulting in collagen disorganization. During this period, VECs may choose to migrate and select new anchoring molecules or carry over fragmented ECM anchors.

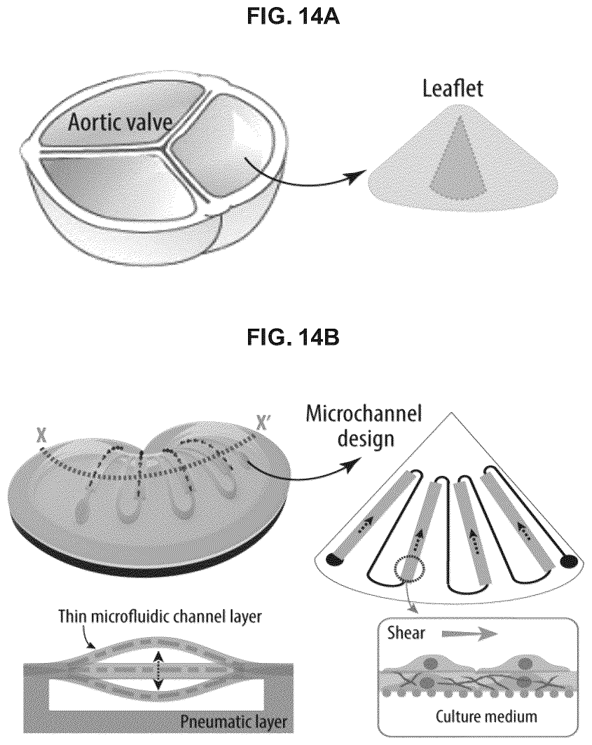

To understand these mechanobiological responses of valvular cells associated with complicated biomechanical environments as well as ECM directional cues, a heart valve organ-chip was constructed using microfluidics and a porous membrane as depicted in FIG. 14A-B. Flexible microfluidics along with an open microchannel concept were used to encase a hydrogel mimicking the multi-layered heart valve scaffold structure composed of collagen and elastin matrices under complex biomechanical stimuli. Using the chamber system depicted in FIG. 14A-B, either a 3D VIC-embedded collagen layer to mimic the fibrosa layer, or a 3D VIC-embedded collagen-elastin layer to mimic the ventricularis can be generated. Both hydrogels were lined with a VEC monolayer. By employing elastin in addition to collagen, a more elastic layer is expected to be created, which will better respond to mechanical forces, with minimal fiber degradation. Cells surrounded by proper ECM are critical for an in vitro heart valve model to recapitulate overall valve structure for studying valvular mechanobiology.

### Development of a Biomimetic Heart Valve Chip to Recapitulate Responses of Valvular Cells in Their Native Environment

Complex mechanical loading conditions govern heart valve function, in both healthy and diseased states. Under degenerative conditions, leaflets are thickened, with eventual loss of function, often associated with increased biomechanical stresses. Thus, heart valve motion and cellular responses are critical factors that need to be understood in order to develop proper therapies for valvular degeneration. It is hypothesized that mechanical and biological properties of a leaflet can be recapitulated in a heart valve organ-chip as described herein and used as a tool to increase the current understanding of valvular mechanobiology.

The disclosed pneumatically controlled cell culture apparatus heart valve organ-chip is comprised of a thin microfluidic chip and a pneumatic actuator, and is the platform for recapitulating biomechanical motion of a heart valve leaflet in vitro. By controlling the flow rate of a microchannel within the thin microfluidic chip, the level of shear stress can be precisely controlled. In addition, flexure/bending stress can be generated by manipulating the pneumatic source on a pneumatic chamber under the thin microfluidic chip. These two main stress components can be controlled simultaneously as well as independently so that precise mechanobiological responses can be obtained using the valve chip.

### Fabrication of the Heart Valve Organ-Chip

A soft-lithography technique was used to fabricate a thin microfluidic chip with microchannels to create the valve chip platform. First, SU-8 molds, containing a simple two-chambered microchannel and a pneumatic chamber, were fabricated to obtain an actuatable microchannel to generate the desired shear and flexure/bending stress profiles. PDMS was then poured into the molds and cured on a 65° C. hotplate. Holes were punched in PDMS microchannel replicas for inlets and outlets and these microchannels were bonded with a porous PDMS membrane. Finally, the thin microfluidic chip was irreversibly bonded to the pneumatic chamber with proper alignment.

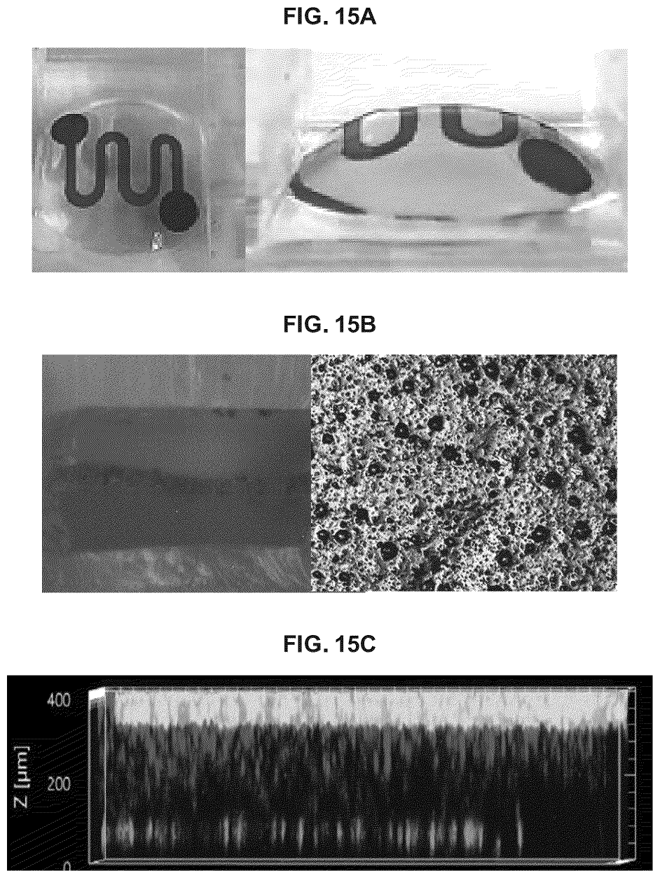

The valve chip comprises two subunits, each of which is a microfluidic chip unit for shear stress control and pneumatic control unit for flexure/bending stress control (FIG. 15A). FIG. 15A shows the successful fabrication of the actuatable valve chip, comprising a thin two-chambered microfluidic chip and a pneumatic control chamber. The porous PDMS membrane was prepared by mixing citric acid with ethanol and toluene thoroughly to dissolve the citric acid into a solution (FIG. 15B). The citric acid mixture was then mixed with PDMS base polymer and boiled at 140° C. for 1 hour until visible crystal formation. The mixture was cooled down and a PDMS curing agent was added. The mixture was then degassed and spin-coated at 2000 rpm to obtain a thickness of about 50 μm. After curing in an oven at 80° C. for 5 hours, the membrane was washed and sonicated in ethanol to remove citric acid crystals and make the membrane porous. Once the thin valve chip was prepared, VIC and VEC cells were cultured in and onto collagen hydrogel, respectively, as shown in FIG. 15C.

Following geometric calculations on the valve chip, biomechanical behaviors (flexural, bending, and shear stresses) are determined. These biomechanical behaviors are determined under various microfluidic chip thicknesses (0.5-2 mm) with a 10-30 kPa pneumatic pressure range, average flow rates (˜90 μL/sec) with unidirectional pulsatile and bidirectional oscillatory flow profiles, and pumping frequencies (1-3 Hz). These different parameters enable the valve chip to generate flexural angles up to 45° and shear stresses up to 60 dyne/cm² for recapitulating biomechanical conditions of aortic valve leaflets during systole and diastole.

During the actuation, the detailed flexural motion of the thin microfluidic chip is measured, and the flow stream and flow profiles are assessed using fluorescent particles on a high-speed camera and highly sensitive flow sensor (Sensorion Inc.) to perform stress-strain analyses. Once mechanical characterization steps are complete, heart valve cells are grown within the microfluidic channel to investigate their mechanobiological behavior under various biomechanical conditions. Flexural angles are known to range between 10° and 50° in aortic valves depending on cusp region. Higher angles towards the free edge correlate to physiological cell phenotypes, whereas smaller flexures correlate to pathology due to increased tissue stiffness. Since the disclosed heart valve chip model does not include a free edge, only local angles are evaluated on chip. Testing a wide range of bends will shed new light on the level of flexural angles that are associated with cell activation. Previous studies have reported that physiological shear levels range from 8-10 dyne/cm² on the oscillatory aortic side of the aortic valve, while averaging 20 dyne/cm² with peaks of 80 dyne/cm² on the ventricular side. In practice, experiments with endothelial cells in culture have shown that applying shear stresses around 20 dyne/cm² poses a cell detachment challenge. However, shear levels up to 10 dyne/cm² have successfully been demonstrated with the disclosed heart valve chip model, and different flow patterns are tested. Once individual effects of flexure and shear are determined, the combination of the two factors is assessed.

A hydrogel consisting of collagen and elastin, and embedded VICs, is injected on the bottom channel of the valve chip to form a biological ECM layer. It is known that fibrosa consists of VEC on a VIC layer made up of a circumferentially aligned collagen-rich matrix while ventricularis layers consist of VEC made up of radially aligned elastin, and embedded VIC. Collagen-elastin gels are prepared as described previously, with or without cross-linking. Briefly, bovine type I collagen solution is mixed with a 5 mg/mL elastin stock solution to yield final concentrations of 3 mg/mL (w/v) collagen and 0-0.6 mg/mL (w/v) elastin, all diluted in DMEM:F12. After adjusting the pH with 1 M sterile sodium hydroxide to 7.5 and crosslinking with squaric acid as needed, the solution is allowed to gel at 37° C. ECM stiffness can be adjusted by varying collagen and squaric acid crosslinker concentrations. The ECM mechanical properties are determined by rheometry prior to its insertion into the chip. Collagen and elastin fiber alignment can be induced by employing a stretching motion through pneumatic layers and fluorescently labelled collagen and elastin are used to visualize their contents in the matrix.

Primary porcine valvular cells are harvested from tissues in tissue dissociation medium (DMEM:F12 medium containing 10% FBS, 1% antibiotics and antimycotics, and 600 U/mL type II collagenase, pH 7.2). Samples are centrifuged to isolate viable endothelial cells. Cells are then resuspended in 1 mL fresh culture medium (as above, without collagenase, with 50 U/mL heparin supplement). After PDMS surface modification, interstitial cells, from either aortic or mitral valves, are seeded within collagen solutions and allowed to gel inside the cell culture apparatus. After gelling, apparatuses are further incubated with a layer of endothelial cells (VECf or VECv).

Initial experiments are conducted with pure VICs and VECs to assess their baseline behavior—fibrosa VECs or ventricularis VECs in monolayer, as well as collagen gel-embedded VICs in 3D. Then, VICs embedded in a 3D matrix are lined with a layer of endothelial cells. Microscopic imaging is performed to check the status of cells in the chip apparatus regularly during the culturing period. In-situ observations of cell morphology and quantification of protein markers that characterize normal fibroblastic (non-degenerative) phenotypes are performed on the multiple valve chips. Furthermore, a long-term culture (4-6 weeks) is performed. When constructs are stable, evidenced by slow growth and minimal VIC activation (vimentin+, TGFβ, and α-SMA to indicate interstitial cell quiescence and a layer of CD31+ endothelial cells), experiments investigating different flexural angles and shear stresses are conducted.

This research demonstrates the ability of the disclosed heart valve chip model to: (1) mimic the motion of a heart valve leaflet; (2) generate 3D ECM patterns for valvular cell culture; and (3) confirm the biomimicry of the valve chip model based on validation with various protein markers. The disclosed valve chip model allows for the generation of various biomechanical environments by tuning flow rate and pressures. These motions are comparable with actual leaflet motions during heartbeat. In addition, the in vitro valve chip models recapitulate 3D ECM structure using a gel with repetitive flexure motion. By adapting flexure motion, collagen and elastin fibers are precisely aligned in the microchannel. While the loss of actuating capability due to repetitive motions under pressure is a potential issue, this can be overcome by adjusting the pneumatic chamber design and using alternative silicon materials with minimal hysteresis. Further, the pneumatic chamber can be printed using 3D printers. Additionally, in case of irregular distribution of VIC at the interface between VIC and VEC, the cell density, geometry, and properties of 3D ECMs are evaluated to optimize culture conditions. These studies will determine and fine-tune the extracellular matrix properties and cell-cell interactions in the disclosed chip system. In addition, side-specific studies of fibrosa versus ventricularis can also show strikingly different responses since the fibrosa experiences oscillatory flow while the ventricularis is exposed to non-oscillatory shear flow. Differences in ECM composition and fiber alignment can also help pinpoint if VECs respond with adaptive or maladaptive remodeling mechanisms. It is known that elastin degradation in the ECM plays a significant function in the progression of degenerative tissue remodeling, with the leaflet curvature being critical to blood flow through the valve and to the rest of the heart. These studies will shed new light into the effect of curvature onto VEC-VIC dynamic responses.

### Evaluation of the Effect of Complex Mechanical Stimuli on the Mechanobiology of Valvular Cells

It has been increasingly recognized that abnormal levels of mechanical stimulation can trigger degenerative processes in valvular tissues and cells. Such degenerative processes result in valvular cell transformation and are mediated by a variety of other signaling mechanisms, converting mechanical stimuli into biochemical responses. For example, different manifestations of valvular degeneration are observed in mitral and aortic valves. Mitral valves typically present myxomatous degeneration of the leaflets or annular calcification. Aortic valves usually present cusp calcification or sclerosis. However, the interacting mechanosensitive pathways governing valvular degeneration have yet to be elucidated.

It is hypothesized that shear, flexure, or a combination of both can induce pathophysiological changes on VECs and VICs cultured on the disclosed heart valve chip. In vitro manipulation of valve constructs is facilitated using a valve chip platform which also allows for repeated manipulations to recapitulate different requirements of different pathologies. These studies will allow for mapping out mechanobiological responses of valvular cells to mechanical stimuli.

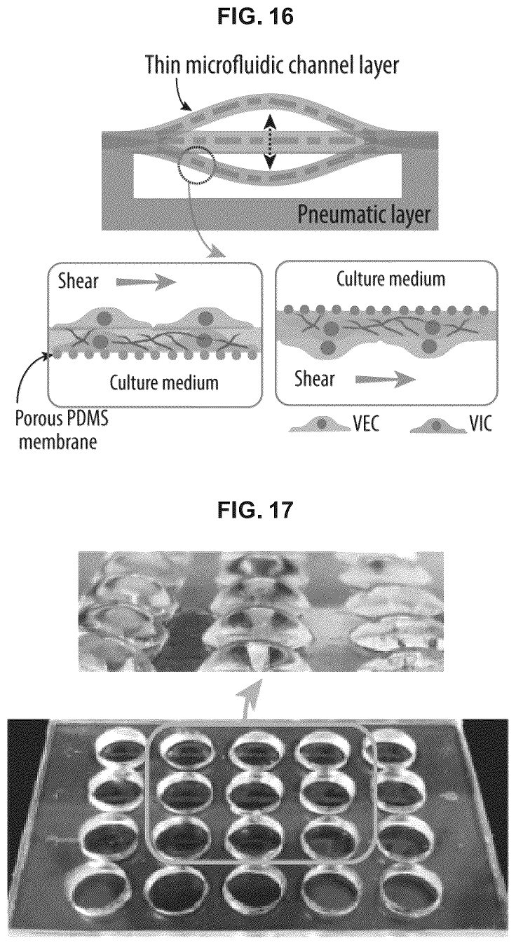

Multiple valve chips were prepared to mimic fibrosa and ventricularis layers. VICs mixed with collagen gel are cultured on the porous PDMS membrane and then VECs are co-cultured on the top of VICs after 24-48 hours to prepare the fibrosa layer (FIG. 16). VICs mixed with elastin gel are cultured over the porous PDMS membrane and then VECs are co-cultured on the other side of the elastin membrane after stabilization of the VIC layer to prepare the ventricularis layer. Endothelialization is verified by immunohistochemistry with an endothelial cell marker, CD31. Valvular constructs on the valve chip system are then stimulated with the application of shear and flexure/bending stresses by controlling flow rates and pressure.

Transcriptome analyses of RNA-seq data are performed to understand the fundamental mechanoresponses. Bulk sequencing is performed to identify differentially expressed genes (DEGs) and determine valvular mechanosensitive genes. DEGs associated with calcification, cell proliferation, angiogenesis, ECM remodeling, cell adhesion, cell migration, and inflammation potentially impacted by mechanical stimuli are investigated. DEGs across different types of mechanical stimuli are identified using RNA-seq. Data analysis techniques are utilized to identify pathways to determine up/down stream regulators of signaling pathways. A complete picture of the transcriptome changes according to various stimuli components is defined and robust biochemical methods are exploited to confirm changes in key gene products using RNA-seq. Four experimental groups of cells (control (C), shear only (S), flexural bending only (F), and both shear and flexure (SF)) are prepared and harvested at Day 2, Day 7, and Day 14. Harvested cells are isolated and prepared for RNA-seq.

RNA is purified using the Qiagen RNeasy Kit. Poly-A containing mRNA molecules are purified from total RNA using the Illumina Stranded mRNA Prep Kit, followed by sequencing with an Illumina NovaSeq 6000. RNA-seq libraries are constructed from 1 μg of rRNA-depleted RNA using the TruSeq RNA library preparation kit following the manufacturer's protocol. RNA is fragmented, templates primed with random hexamers and PCR-synthesized. Once surveys of transcripts are complete, a signaling map is built with Ingenuity Pathway Analysis (IPA) to determine pathways that are activated. RNA-seq reads are trimmed with Cutadapt, aligned to the pig genome reference (Sscrofa11.1) with STAR (Spliced Transcripts Alignment to a Reference) aligner, and transcript abundance estimated with feature Counts and RSEM.61 DEGs (between combination of mechanical stimuli) is performed and evaluated using the DESeq2 package. Lastly, a comprehensive set of quality control metrics is calculated using Picard and Bioconductor tools with standardized QC report assembled with multiQC. These include sample level principal component analysis, and read gene feature mapping statistics to assess and remove outlier sample datasets from the analysis. DEGs, as defined as those with an absolute log 2ratio>1 and adjusted p-value of <0.05, are subjected to a complete pathway and ontology term analysis using the IPA platform.

With these data, it is possible to analyze and identify pathways, understand if the pathways intersect or work in parallel, and determine which upstream regulators are involved. High-quality mRNA is used in the construction of libraries using Illumina Design Studio, particularly focusing on TGFβ and serotonin transcriptional activation. A major goal is to identify pathways characteristic of physiological versus pathological states. After determining key transcripts among all DEGs during the in-silico analysis, qPCR and immunoblotting are performed to validate the mRNA and protein levels of samples of transcripts identified in each group. By conducting parallel studies with aortic and mitral valvular cells, these studies allow for the characterization of the central differences in degenerative mechanisms leading to myxomatous degeneration (aVIC predominant) versus calcification (obVIC predominant).

Experimental groups of cells (control (C), shear only (S), flexural bending only (F), and combined shear and flexure (SF)) are prepared for each culture type (pure cultures of VECf, VECv, VICs, as well as co-cultures of VECv-VICs and VECf-VICs) and harvested at Day 2, Day 7, and Day 14. Microscopic imaging and other biochemical analyses then follow. Collagen and elastin alignment are investigated for each experimental group using fluorescence staining. Experiments include different types of cultures (pure VICs, pure VECs of either side—VECf and VECv—and VEC-VIC co-cultures), each undergoing a series of shear stresses (0-20 dyne/cm²), a series of flexures (0-50° flexural angles) and a combination of the two. Endothelialization is verified by immunofluorescence with an endothelial cell marker, CD31. Validation of endothelial cell purity is accomplished by CD31+ cell magnetic separation. Endothelial cells are incubated for 8 hours in a CO₂ humidified incubator to allow initial adhesion. After, the cell culture medium is replenished until growth reaches confluence. Valvular constructs on the valve chip system then undergo stimulation regimens applied for 24 hours, and at the end of tests, microscopic imaging and other assays are performed.

Characterization of cell transformation is conducted using cell immunostaining and microscopy in situ, paired with RNA sequencing to quantify transcripts linked to cell activation in each condition tested. Fluorescent microscopy evaluations provide validation, visualization, and a quick assessment of outcomes, whereas RNA sequencing provides a complete transcriptomic profile as cells respond to the mechanical environment. Immunofluorescence is performed on constructs with markers of cell type. The following pairs of endothelial and mesenchymal protein markers are used: CD31, vWF, TGFβ, and β-catenin. All treatments undergo double immunofluorescence experiments (at least one endothelial and one mesenchymal marker) for verification of unexpected cell phenotype change. In addition to experimental controls, all immunostaining is performed with standard negative controls. Four random regions of interest are chosen randomly for analysis for each imaging experiment. Fixed cell samples are analyzed in a confocal microscope for a more comprehensive 3D view of constructs. Cell aspect ratio and order parameter are also evaluated to account for possible fibroblast versus cuboidal phenotypes. ImageJ is used to compute cell area. Immunofluorescence results are used to determine the presence of quiescent (vimentin, discoidal domain receptor 2), myxomatous (α-smooth muscle actin, embryonic smooth muscle myosin, and matrix metalloproteinases 1 and 13) or calcific markers (bone morphogenetic protein 2, runt-related transcription factor 2 and lipoprotein receptor-related protein 5). Next, markers of TGFβ signaling are profiled in all experiments. Cell phenotypes across each stimulation type, as well as with those of normal aortic valve leaflets, are evaluated using two-way analyses of variance (n=6 for each group). If necessary, experiments are started with transformed VICs (aVICs or obVICs) to test for phenotype reversal by mechanical stimuli.

High Throughout Mechanistic Assessment of Valvular Cell Mechanotransduction Pathways that Regulate Pathophysiology

An understanding of mechanically regulated cellular processes is dependent on elucidating the mechanisms of biological responses. By combining the therapeutic targets of important mechano-regulated pathways with predicted transcriptome expression profiles using a high-throughput platform, pathways that are suppressed or activated can be predicted under a wide range of biomechanical conditions. These high-throughput studies will allow for the determination of a mechanically regulated major pathway so that a pharmacological inhibitor may reverse a diseased phenotype. By miniaturizing and testing mechanical conditions in a high-throughput format of the disclosed heart valve chip, the specific conditions that enable pathophysiological signatures from valvular cells during in vitro culture can be determined. It is hypothesized that by systematically employing inhibitors (pharmacologic or siRNA-based) in multiplex high-throughput chips, mechanically regulated pathways are able to be identified which control the fate of valvular cells. For example, a mechanism for preventing myofibroblastic or osteoblastic activation can be identified.

Select mechanical environments most inducive of pathological transformation are chosen, and pharmacological or siRNA inhibitors targeting TGFβ—a well-known mediator of cell activation and osteoblastic transformation—are tested to assess cell phenotype. The inhibition experiments determine if pathological transformation pathways are unique (dependent uniquely on TGFβ or others), and identify routes of reversal of cell transformation, which can lead to the identification of drug targets. Different signaling nodes of TGFβ inhibition are evaluated with different inhibitors and concentrations as described below.

A heart valve chip array was fabricated to transform the planar surface with diverse flexure bending using a precise pneumatic control system (FIG. 17). The array was comprised of a pneumatic chamber for actuation and a 250 μm thin PDMS flexible membrane embedded with a microfluidic channel for culturing valve cells and supplying medium to the cell using a syringe pump. This array chip was evaluated to determine control parameters for the proposed level of flexure bending and cyclic motion. There was almost no hysteresis detected during the cycle motion after a week. Based on the success of these studies, this array is expanded to a 96-well plate scale format, along with modular pneumatic and fluidic control.

Studies are performed to test three response groups undergoing TGFβ inhibition: (1) most physiological or high qVIC counts, with no evidence of activation on transcriptome; (2) osteoblastic activation or high obVIC transformation; and (3) myofibroblastic activation or aVIC transformation. These experiments use TGFβ1 siRNA (s14056 Sigma,), as well as small molecule selective inhibitors of TGFβ. Additional experiments are also performed to investigate the inhibition of other pathways besides TGFβ1. As it is common with TGFβ pathway mechanisms, downstream targets will include SMADs, Snail, and MMPs. A primary goal is to create a range of constructs with different levels of mechanically induced degeneration, dependent or independent of TGFβ signals.

Mechanical stimuli are varied based on cell phenotype results. Drug effects are verified by quantification of TGFβ signaling from cells after 24 hours of exposure, in addition to live fluorescent imaging of different regions within the heart valve chip. Characterization of cell transformation is conducted by cell immunostaining and microscopy in situ, paired with RNA sequencing to quantify transcripts. Fluorescent microscopy evaluations provide quick visible assessment of outcomes, whereas RNA sequencing provides a complete transcriptomic profile as cells respond to the inhibitors applied. Immunofluorescence is performed on constructs with specific markers for each cell type. The following pairs of endothelial and mesenchymal protein markers are used: CD31, vWF, TGFβ, and β-catenin. All treatments undergo immunofluorescence double stains (at least one endothelial and one mesenchymal marker) for verification of unexpected cell phenotype change. Additional tests of inhibitor concentrations may also be required.

### Example 6

### Interface and Base Components for High-Throughput Array Chips

FIG. 18A-B show an example cell culture apparatus illustration depicting a high-throughput (HT) 96-well array configuration comprising a plurality of cell culture chambers as described herein. The high-throughput format may comprise any one or any combination of the various cell culture chamber embodiments or flexible membrane embodiments as described herein to generate a high-throughput cell culture apparatus. FIG. 18A shows that with a 96-well HT organ-chip configuration and independent pneumatic control ports, various parameters (e.g., curvature, flexure, bending stress, shear stress, etc.) may be simultaneously or independently controlled to mimic microphysiological environments precisely. FIG. 18B shows a side view of the HT organ-chip of FIG. 18A.

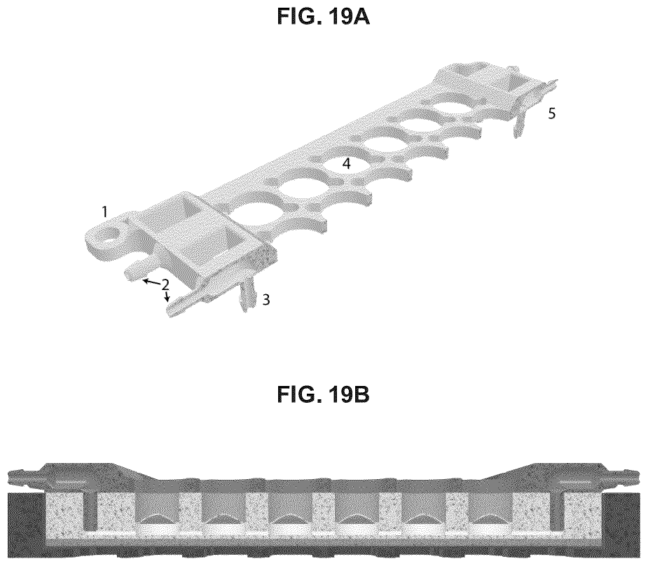

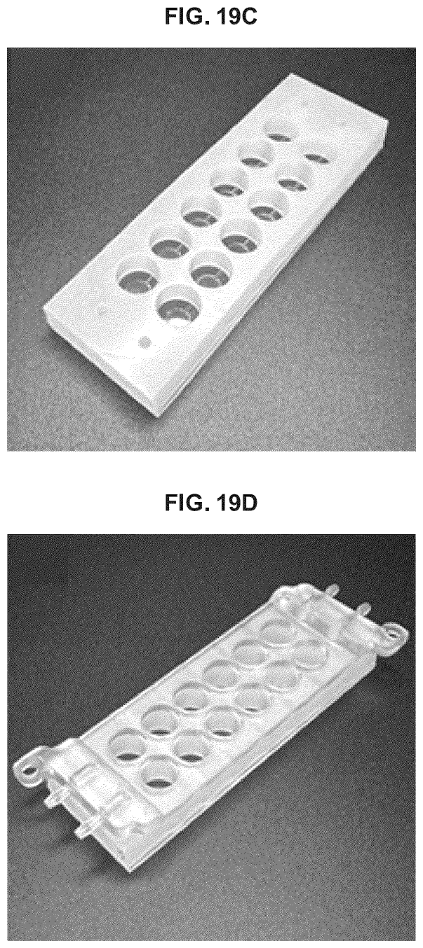

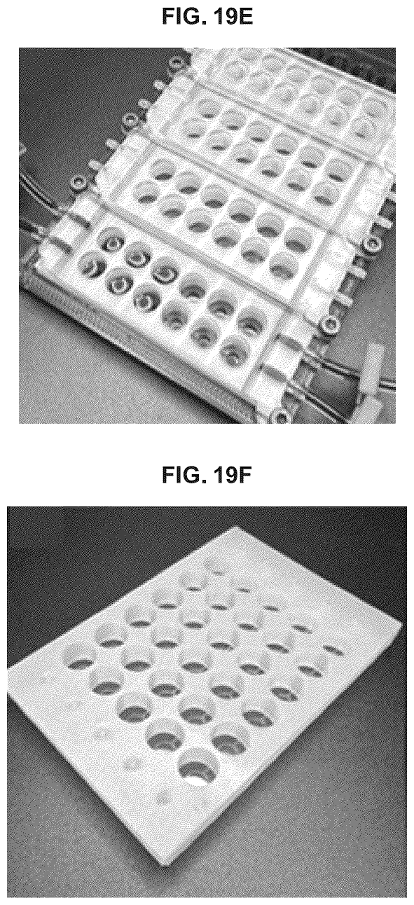

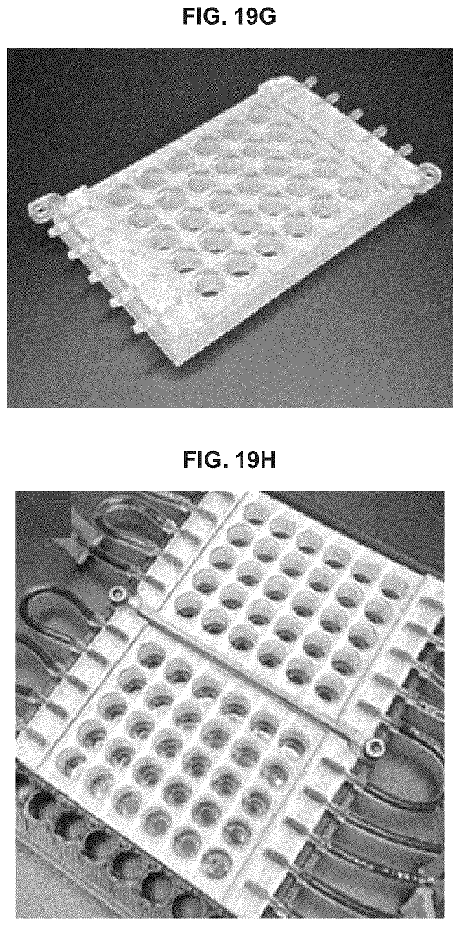

In some embodiments, disclosed high-throughput chip platforms may include a two-piece integrated interfacing manifold system that connects the microfluidic system to the pneumatic or liquid pressure generation that creates the curvature membrane balloons (FIG. 19A-H). The interface component was made up of two pieces, a plastic piece that interfaces with each microfluidic chip, and a matching base that holds the microfluidic chips and plastic pieces into place. The interface may help to allow for control of the membrane balloon size across multiple rows of wells without the need for complicated external tubing manifolds. Additionally, the integrated interface piece allows for a smaller overall footprint, allowing for the system to match existing 96-well plate devices.

The base of the interface matches the existing 96-well plate footprint to ensure that this platform can be used with the existing laboratory equipment designed for use with conventional well plates. The height of the overall system is a combination of the microfluidic chip depth and the plastic microfluidic interface piece and matches that of a conventional 96-well plate format. Additionally, some pieces of equipment may hold the 96-well plates through a notch on one of the corners, which was also replicated in the base piece. The upper plastic piece may have barbs for attachment to the microfluidic chip as well as tubing for connecting to the balloon pressure source (e.g., pneumatic actuator). Each connection to the pressure source may be isolated so that different rows of wells can be independently actuated at different rates and at different inflation amounts. Both parts of the interface were fabricated using 3D printing, and the top plastic piece was specifically fabricated with SLA printing to allow for the precision necessary for the internal connections and channels. The base piece was printed with FDM printing methods. However, these fabrication methods are not limiting or instrumental to the production of this platform and have only been utilized for rapid prototyping in this example. For example, each part of the manifold may instead be produced using injection molding for high scale manufacture of inexpensive products.

FIG. 19A-H show an exemplary cell culture apparatus array for high-throughput applications. FIG. 19A shows an exemplary interface component for a 12-well layout that connects the described cell culture chamber arrays to a pneumatic actuator by interfacing with the microfluidic chip. 1—Point to secure each individual microfluidic chip to a base and the interface against the chip itself. 2—Barb fittings for connections to outside pneumatic and liquid pressure sources. 3—Barb connections for interfacing with the microfluidic chip itself, sized to match a 96-well plate footprint. 4—Cutouts/apertures to allow access to each individual cell culture chamber/well by 96-well based equipment and for imaging applications. Two smaller cutouts, diagonally opposed, are used to allow for different cell seeding methods. 5—Matching barbed features on the other end of the interface to be used as an outlet during initial filling. FIG. 19B shows a side view image cut through the first set of wells showing the entire integrated apparatus with a microfluidic chip layer comprising a plurality of cell culture chambers supported by a top interface component and a bottom base component, where the plurality of cell culture chambers is connected to an external pneumatic actuator pressure source through the upper interface component. FIG. 19C-H show the high-throughput system in different states of assembly. FIGS. 19C and 19F show 12-well and 30-well plate compartment chip configurations, respectively. FIGS. 19D and 19G show the 12-well and 30-well plate compartment chips with an interface component mounted on top. FIGS. 19E and 19H show the 12-well and 30-well plate compartment chips with an interface component mounted on top and a bottom base component, where the chips are connected to an external pneumatic actuator pressure source through the upper interface component. The 12-well chip in FIG. 19C can be used in 12-, 24-, 36-, or 48-well configurations, and the 30-well chip in FIG. 19F can be used in 30- or 60-well configurations. Larger configurations of the 12-well and 30-well chips are also possible beyond these sizes.

The disclosed high-throughput platform has multiple benefits over similar microfluidic systems and interfaces. First, the overall system is designed to be compatible with existing 96-well plate laboratory infrastructure. This is unique to the disclosed systems and provides high functionality. Second, the fabrication of all components is easily scalable, leading to a low-cost platform to compete with the existing 96-well plate infrastructure. Additionally, the system is designed to be modular using chips with either 12 or 30 wells, depending on the base used. The system can be used with any number of chips based on the requirements of the experiments. Lastly, the actuation of the wells can be used to induce fluid motion in the wells, which aids in mixing and increases reaction speed to shorten assay times.

### Example 7

### Functional High-Throughput Lung Organ-Chip Model

### Materials and Methods

### Fabrication of High-Throughput Compartment Array Chip and Experimental Preparation

To fabricate the device, polydimethylsiloxane (PDMS) (Sylgard 184, Dow Inc.) with a 10:1 ratio (base:curing agent) was poured onto a 150 mm petri dish and allowed to cure overnight at 70° C. in an oven. After curing, PDMS was cut with 6 mm diameter holes to create the top well layer to create cell culture chambers (FIG. 20). A PDMS sheet (BISCO HT-6240, Rogers Corp.) with a thickness of 250 μm was used to create the microfluidic channel, and a PDMS membrane having a thickness of 100 μm was used to create the curvature and dynamic motion. The fluidic channel was designed with AutoCAD (Autodesk), and a PDMS sheet was cut with CAMM-1 GX-24 24″ Vinyl Cutter (Roland DGA Corp.). FIG. 20 shows a cross-sectional view of the device. The clear PDMS microfluidic channel layer with PDMS membrane was plasma bonded together and then was oxygen plasma bonded with a glass slide. The top well layer was then bonded on the top of the PDMS membrane layer by plasma bonding. After successful cell culture, PVC tubing (B-44-4X, Tygon) was connected from a programmable pneumatic actuator syringe pump device (Fusion 200, Chemyx) to the inlet of an interface component that connects to the fluidic layer to make a dynamic motion.

Inventor (Autodesk) was used to design individual components of the high throughput dynamic cell culture system. A chip holder matching the footprint of conventional 96-well plates was designed first with regions to hold individual microfluidic chips and was printed using a filament-based 3D printer (Lulzbot Taz Pro). This made the system modular and capable of including anywhere between 12 and 60 wells, depending on the number of chips and the layout used. A pneumatic interface component as described above was also designed and printed using a resin-based 3D printer (Form 3) due to its ability to create high-resolution features, which is necessary for an integrated barb interface. The compartment chips are shown in two sizes (12-well and 30-well) in FIGS. 19C and 19F. The two sizes correspond to commercially available glass slides that were used as supporting backing for the microfluidic chips themselves. Increasing the usable wells from 60 to 96 is also possible with further development and purpose-built equipment to produce the required tolerances and custom glass to fit more wells while maintaining the spacing of conventional well plates.

### Angle Measurement Based on the Volume Injection and Determining the Concave Volume

To determine the angle based on the different volumes, the pneumatic actuator syringe pump was used to inject the different volumes, and a microscope was used to capture images of volume change. ImageJ was used to calculate the angles and the data were quantified to calculate the trendline equation. The concave volume of the membrane in response to applied water volume was calculated using the linear strain equations and trendline equation from physical angle vs. volume experimental data.

### Computational Modeling to Verify Strain Regions

Breathing motions of the PDMS membrane were modeled with finite element analysis (FEA) in COMSOL Multiphysics (COMSOL Inc.). The membrane was designed as a cylindrical disc with a radius of 2 mm and a thickness of 0.1 m, and a hyper-elastic material model with PDMS properties was used to understand the change of the total displacement in the single balloon.

### Membrane Coating of Polydopamine and Fibronectin

To increase cell adhesion onto the PDMS membrane, the membrane surface was coated with polydopamine and fibronectin. 5 mg of dopamine hydrochloride (H8502, Sigma-Aldrich) was added to 50 mL of 1.0 M Tris HCl pH 8.5 (MB-027-1000, Rockland Immunochemical) to make 0.01% (w/v) concentration polydopamine. 50 μL of 0.01% polydopamine was applied to the membrane for 24 hours. After polydopamine coating, the membrane surface was washed twice with DI water to clean unattached polydopamine molecules. Afterwashing, 1 mg/mL of fibronectin (F0895, Sigma-Aldrich) was diluted with Gibco™ PBS, pH 7.4 (10010-031, Thermo Fisher Scientific Corp.) in a ratio of 1:100 and coated onto the membrane surface for 2 hours at room temperature after washing the membrane surface. The polydopamine-fibronectin-coated chip was stored at 4° C. until use.

### Cell Culture Protocol

A concentration of 1×10⁶ cells/mL of A549 cells, a human lung adenocarcinoma type II cell line (CCL-185™, ATCC), was maintained in 500 mL of Gibco™ Ham's F-12K (Kaighn's) Medium (21127022, Thermo Fisher Scientific Corp.) supplemented with 50 mL (10%) of Gibco™ Fetal Bovine Serum (FBS), qualified, One Shot™ format, New Zealand (A3160902, Thermo Fisher Scientific Corp.), and 5 mL (1%) of Gibco™ Penicillin-Streptomycin (10,000 U/mL) (15140122, Thermo Fisher Scientific Crop.) in the compartment chip. Once the cells were confluent, cells were dissociated with 0.25% Trypsin-EDTA for 5 min in the incubator. After dissociating the cells, single cells were prepared and suspended in the corresponding medium at a concentration of 1×10⁶ cells/mL, and 40 μL of the cell suspension was loaded into each PDMS well. The cell-loaded devices were left undisturbed for 2 hours to allow cell adhesion. After 2 hours of cell adhesion, PDMS wells were washed with PBS and 100 μL of the medium was added to each PDMS well. The cells were cultured in a submerged condition and the F-12k medium was changed every 24 hours until a monolayer was formed for 4 days. After forming the monolayer, tubing was connected from the fluidic inlet to the pneumatic actuator syringe pump to induce a breathing-like motion.

### Air-Liquid Interface Using Dynamic Motion and a Syringe Pump

A dynamic motion was actuated using the built-in function of the Fusion 200 pneumatic actuator syringe pump. In order to mimic the normal respiratory breathing rate of 12 breaths per minute, the pump was set to an actuation frequency of 0.2 Hz. After setting the frequency, volume, and flow rate in the syringe pump, the dynamic motion was actuated on the dynamic array device.

To create an air-liquid interface, a minimum amount of medium volume needs to be identified to make the membrane surface expose to air and submerge back into the medium. Due to the meniscus formation in the well, five different volume amounts were added to the well to identify the maximum exposure to the air.

### Immunofluorescence Staining and Imaging

Cells were washed two times with PBS and fixed with 4% PFA for 10 min. The cells were washed again with PBS three times for 5 min and treated with a permeabilization solution (94.7% PBS: 5% FBS: 0.3% Triton X-100) for 1 hour at room temperature. After permeabilization, the cells were treated with Phalloidin, Tetamethylrhodamine B isothiocyanate (P1951, Sigma-Aldrich) (1:50), Anti MUC5B primary antibody (HPA008246, Sigma-Aldrich) (1:50), 2% BSA, 0.2% Triton X-100 overnight at 4° C. On the next day, the cells were washed with PBS 3 times for 5 min. Afterwashing the cells, the secondary antibodies (Anti-rabbitAlexa Fluor488 (ab150077, Abcam) (1:500), Anti-rabbit Alexa Fluor 594 (A-11012, Invitrogen) (1:500), Anti-mouse Alexa Fluor 488 (SAB4600388, Sigma-Aldrich) (1:500)), and 2% BSA+0.2% Triton X-100 were added for 1 hour at room temperature in the dark. The cells were washed again with PBS 3 times for 5 min. Lastly, Hoechst 33258 (H3569, Invitrogen) (1:3000) was added in the well and cells were imaged using a Nikon Eclipse Ti-E inverted microscope. Fluorescence intensity was quantified using ImageJ.

### Example 8

### Results and Advantages of the Functional High-Throughput Lung Organ-Chip Model

These studies describe a scalable and high-throughput lung organ-chip model system for investigating mechanobiological effects and drug responses under dynamic motion. Dynamic cell culture is crucial for studying the mechanical changes in cells and tissues under physical stress in mechanobiology. Thus, there is a need to improve high-throughput organ chip systems for investigating mechanobiology and drug responses. The disclosed lung chip model system is a 96-well-based, high-throughput triaxial dynamic cell culture platform with customizable hydraulic and pneumatic pressure. This model allows user-controlled conditions and compatibility with automated equipment. The results show that the system has the ability to mimic the normal breathing motion of the alveolus, which is a 4% linear strain from the original size and is useful for mechanobiology and drug screening. Compared to the flat cell culture, cell alignment on both static and dynamic curvatures of cell cultures was observed along the circumferential strain, but was random at the center of the membrane, as observed from F-actin expression. While the high-throughput and dynamic organ chip was successfully validated for lung epithelial recapitulation, this high-throughput model system may also be used with other organ chip systems (e.g., cornea epithelium, heart valve) where curvature and dynamic motion are required. This system may be used to investigate parametric drug screenings.

A 96-well microplate is one of the most commonly sized well plates used in clinical diagnostic testing and analytical research laboratories due to its high-throughput screening, compatibility with automated equipment, and experimental effectiveness and efficiency. Numerous microplate readers exist to analyze and diagnose biological, chemical, biochemical, or physical events from samples in these well plates. Nevertheless, there is a lack of functionality to study the effects of dynamic cell culture with triaxial heterogeneous mechanical stress to recapitulate the mechanical motion of organs such as the alveolus of the lung, the cornea, the heart, etc., on the currently available 96-well plate. To address this issue, organ-on-a-chip (OOC) has been developed to actuate different types of mechanical stress onto cells. Furthermore, OOC can actuate heterogeneous mechanical stress on both static and dynamic curves on the cells to improve the physiological relevance of the microenvironment being studied by matching in vivo physical conditions based on microfluidics. However, due to the complexity of the design and fabrication process, developing a high-throughput OOC system is limited and challenging.

Heterogeneous mechanical stress on a static curve can affect cell behavior in various ways, including changes in cell proliferation, differentiation, and gene expression. The curvature of the culture surface can influence the organization of the cytoskeleton, which in turn affects the mechanical properties of the cell and its ability to interact with its surroundings. The curvature can also alter the distribution and availability of signaling molecules and nutrients, affecting cellular signaling pathways and metabolism. Thus, cells can respond differently to different types of curvatures, and the curvature of the culture surface can affect the outcome of experiments designed to study cellular behavior. For example, cells grown on a concave surface may behave differently than cells grown on a convex surface, and these differences can impact the interpretation of experimental results. This type of static curve cell culture can be important to study the cornea and cochlea, for example, due to their curvatures.

Heterogeneous mechanical stress that is dynamically varied can also affect cell behavior. The cyclic changes in the curvature of the culture surface can stimulate the cell to respond and adapt, leading to changes in its behavior and function. Dynamic cyclic curve cell culture can provide a unique way to study cellular responses to mechanical stimuli, an essential aspect of cell biology. For example, researchers can use this type of culture to study how cells respond to changes in mechanical signals and how these responses can impact cellular behavior. This dynamic curve cell culture can be essential to study lungs, heart, etc., due to their cyclic dynamic motion.

In this study, a 96-well-based high-throughput triaxial dynamic lung cell culture system was developed to improve on the deficiencies and weaknesses of current 96-well plates and OOCs. There are many advantages of the disclosed high-throughput chip system. First, this system can be manipulated with both dynamic and static experimental cases based on the control of the pressure. Due to its material property of polydimethylsiloxane (PDMS), both pneumatic and hydraulic pressure can be introduced to the system. Moreover, dynamic motion can be easily controlled depending on the control of pressure, flow rate, and frequency of the pressure control instrument. Second, the number of samples can be easily controlled depending on the dimensions of the chip design. To be specific, the pressure interface can be easily modified based on how the user connects the pneumatic ports, which can increase or decrease the number of samples at the same time. Third, this system can also be applied to existing automated equipment due to it matching the size of a conventional 96-well microplate.

To demonstrate this system, the effects of dynamic motion on alveolar epithelial cells were investigated because of the natural characteristic of the breathing motion. Hydrostatic pressure was introduced using a pneumatic actuator into the system to recapitulate the deformation of the alveolar wall base membrane due to the pneumatic pressure. Furthermore, dynamic and static cell cultures in the presence of an air-liquid interface were compared by measuring MUC5B expression, which is a marker of surfactant generation, in each case. Moreover, the mechanobiological effects of alveolar epithelial cell morphology on actin variance were examined, along with the net displacement caused by triaxial stretch motion. By developing this high-throughput system, numerous experimental cases can be easily setup and manipulated to compare with control for drug screening and mechanobiology studies.

FIG. 20 shows a cross-sectional view of exemplary compartment chips. White PDMS was used for defining culture well chambers and for the inlets/outlets for the pneumatic interface. A clear PDMS sheet was used to culture cells, and the mechanical motion was applied from the pneumatic interface. A glass slide was the base of the chip, which holds and supports the entire system. FIGS. 19E and 19H show the high-throughput system in 48-well and 60-well configurations, respectively. Both configurations match conventional 96-well plate platforms for integration into existing testing equipment. Also, dynamic motion and curvature can be modulated differently across each row of six wells to increase the throughput of experiments. Additionally, the dimensions of the well or the number of wells could be simply modified to match the needs of a particular experiment. FIGS. 19E and 19H also demonstrate how this high-throughput system could control the number of experimental cases based on how the tubing is connected. The 12-well chip may be used in 12-, 24-, 36-, or 48-well configurations, and depending on how the user connects the tubing, the sample number can be modified. The 30-well chip may be used in 30- or 60-well configurations., and depending on how the user connects the tubing, the sample number can be modified. Larger configurations of the 12-well and 30-well chips are also possible beyond these sizes.

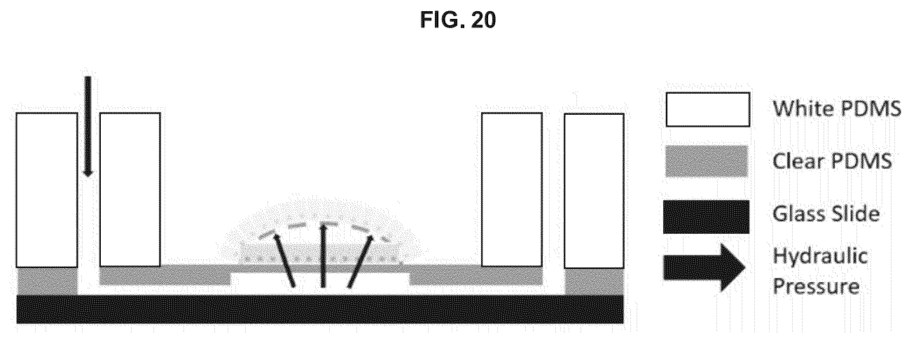

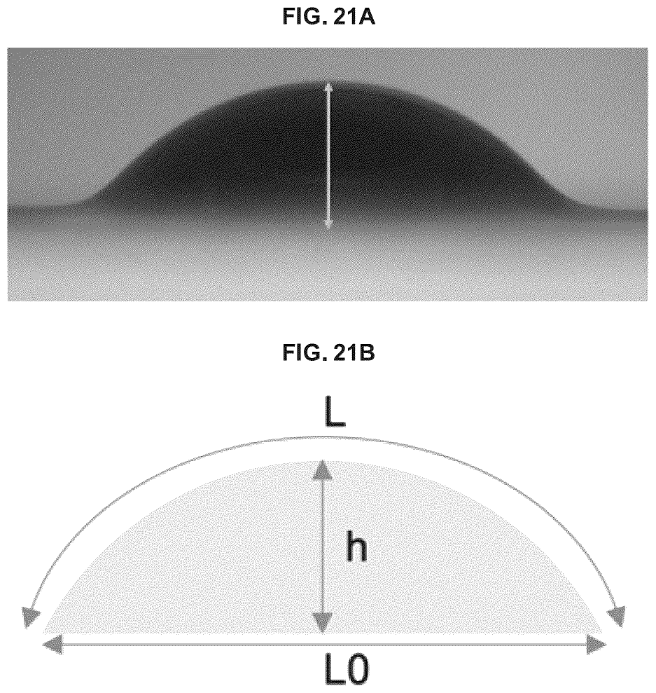

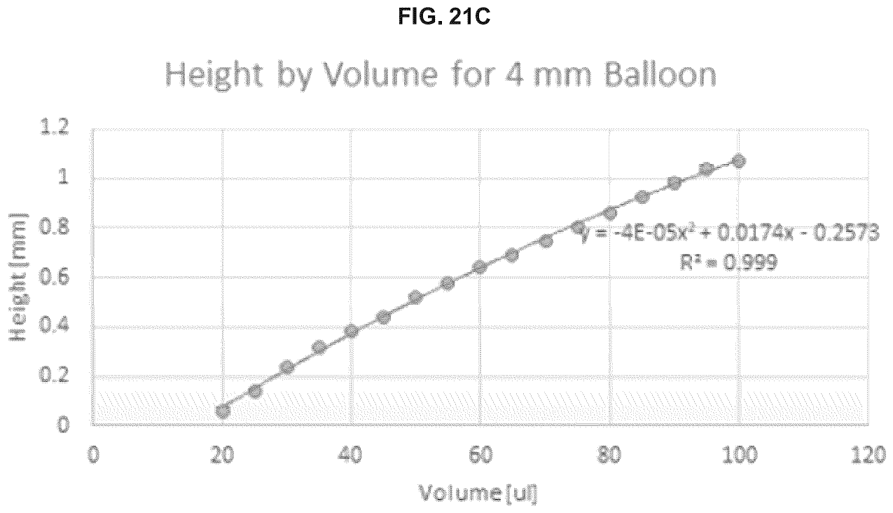

FIG. 21A shows the change in height after adding 100 μL into the balloon. ImageJ was used to measure the height of the balloon with different volumes from 0 to 100 μL. FIG. 21B shows the linear strain calculation. FIG. 21C shows the change of height by adding 5 μL each until 100 μL is present in the balloons. From 0 to 20 μL, the height was not able to be measured due to the insufficient volume to inflate balloons. Also, the curvature was not able to be mimicked due to the size difference between the alveolus and the balloon. Thus, initial curvature was calculated by simply increasing the size of the sphere. Based on the linear strain equation, height (h) was calculated as 535.898 μm, which required a minimum volume of 83 μL from the trendline equation to develop the initial curvature. To mimic the 4% linear strain from the initial curvature, h was calculated as 740.772 μm, which required 115 μL total. Therefore, 32 μL was infused and withdrawn from the initial curvature to make a 4% linear strain, mimicking normal breathing.

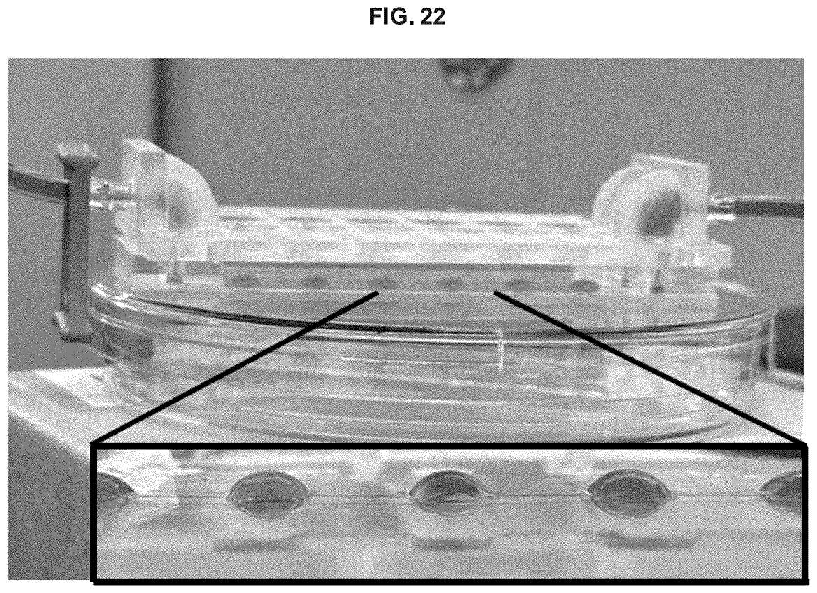

FIG. 22 shows a mechanical motion test on the compartment chip. To better visualize the mechanical motion, the well was removed. From this test, it was confirmed that the mechanical motion in series is consistent due to the hydraulic pressure and the incompressibility of water at low pressures. Both static and dynamic motion were performed in the incubator to observe the consistency of the motion on the chip, and each case was successful without any leakage. Slide clamps held the hydraulic pressure on both the static and dynamic motion experiments. For the static motion experiment, two clamps were used to hold the pressure on both the inlet and outlet. For the dynamic motion experiment, only one clamp was used to hold the pressure on the outlet.

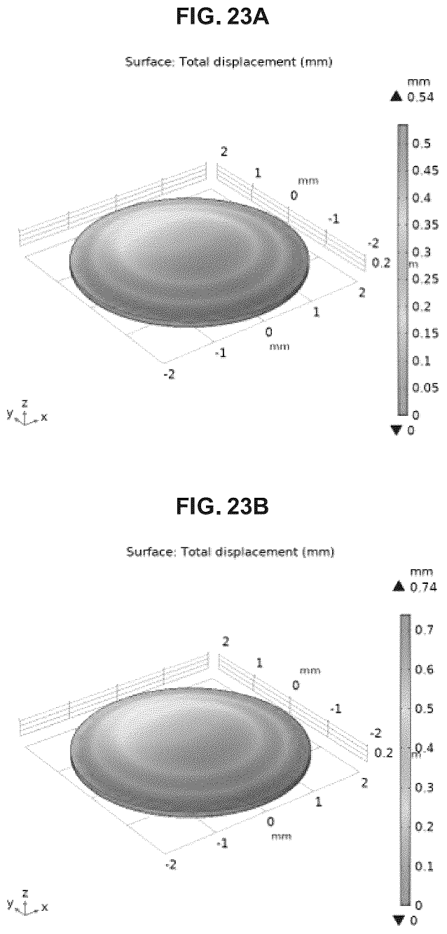

FIG. 23A-B show the finite element analysis (FEA) of the membrane balloon. From the calculation in FIG. 21C, a pressure of 884.7 Pa was set to make the initial curvature height of 535.88 μm. A pressure of 1884 Pa was set to make the 4% linear strain curvature from the initial curvature height of 740.77 μm. Using the same pressures, the first principal strain was analyzed for both the initial curvature and the 4% linear strain curvature. To compare with the experimental value of the pressure of inflating the balloons, this computational analysis was a fundamental step to consider the hydraulic pressure in the system.

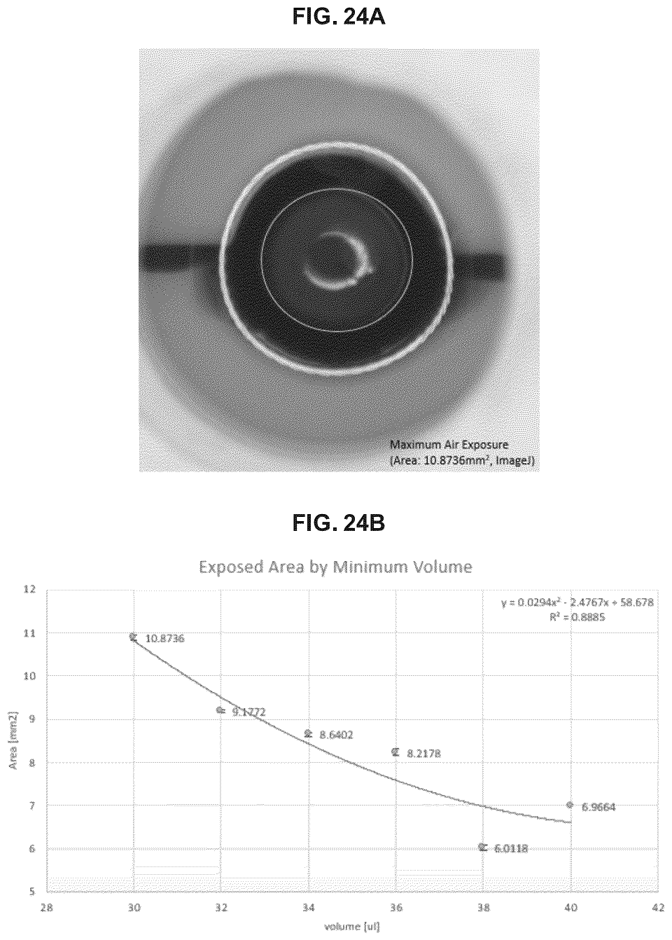

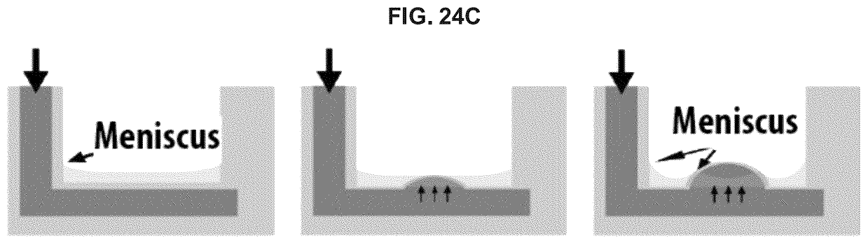

FIG. 24A-C show the minimum volume of media to test for air-liquid interface formation. The air-liquid interface culture is an important feature for respiratory research due to its physiological properties. Therefore, the control of the meniscus can be important in forming an air-liquid interface. A minimum volume of cell culture media was identified by introducing different volumes of the culture media to the well to create maximum exposure of the balloon. From this experiment, 16 μL showed the most air exposed with an area of 5.5032 mm² on the 6 mm diameter well. Depending on the diameter of the well, the minimum media volume should be carefully considered to control the meniscus formation.

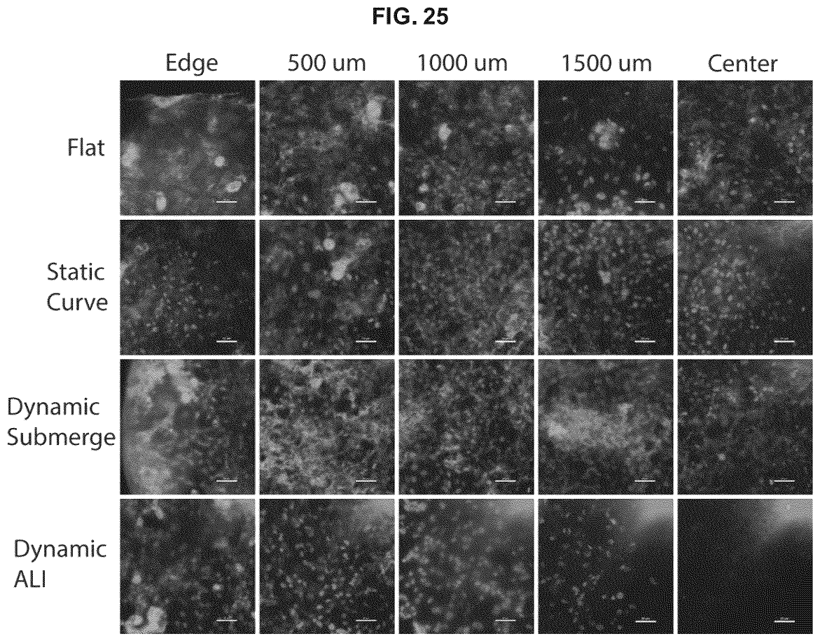

FIG. 25 shows images of F-actin (phalloidin, red), Collagen Type 1 (green), and Hoechst 33258 (blue) staining for each case at the edge, 500 μm, 1000 μm, 1500 μm, and center of the flexible membrane balloon. Due to the mechanical stress on the cells, cell density was increased towards the edge, and cells were stretched between 500 μm and 1500 μm, where the strain was increased the most on the curvature.

The flat surface of PDMS was to mimic 96-well plates to compare with a static curve, dynamic submerge, and dynamic submerge air-liquid interface (ALI). Compared to the flat, the static curve submerged, dynamic submerged, and dynamic submerged ALI showed noticeably denser cell densities. Also, stretching of the F-actin structure was observed between 500 μm to 1500 μm, where the maximum stretch is applied on the balloon. On the center area of the air-liquid interface, cell death occurred due to the insufficient cell culture media supply. Therefore, the mechanical motion should have been modified to make sure the media supply was still on the center area. Although the air-liquid interface was unsuccessful in this experiment, it was dimly observed that the cell morphology changed in the center area where the cells were exposed to the air. The cells were more rounded and tightly bound together. Moreover, on the center of the membrane, cell alignment was random by observing F-actin expression due to the physical property of the balloon where there is no stress occurring. However, cell alignment was observed along with the circumferential strain on the middle area and edge of the membrane.

Furthermore, epithelial-to-mesenchymal transition (EMT) is an important process in lung epithelial cells, driving tissue remodeling and contributing to various lung diseases such as pulmonary fibrosis and lung cancer. Collagen type 1, which is a major component of the extracellular matrix, is upregulated during EMT and has been shown to contribute to fibrotic changes and tumor progression in the lung. Collagen type 1 expression was negative, indicating that EMT transition did not occur during the mechanical motion. These studies confirmed the potential use of the triaxial dynamic culture array chips in enabling the mimicry of the alveolus respiratory motion, and further demonstrate that the disclosed array chips are useful devices for studying the mechanobiology of alveolar epithelial cells with a high-throughput system.

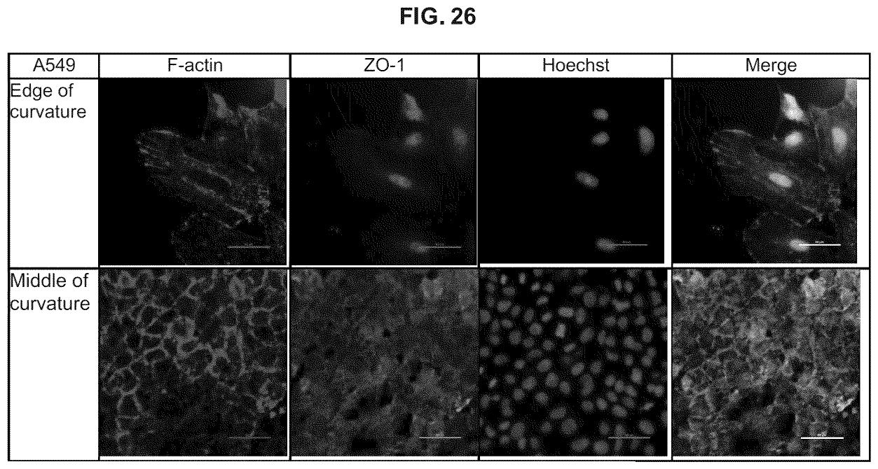

Furthermore, FIG. 26 shows F-actin (red), ZO-1 (green), and Hoechst 33258 (blue) immunofluorescence staining in cultured A549 lung carcinoma epithelial cells at the edge of curvature and the middle of curvature of the flexible membrane balloon.

### Example 9

Curvature Array Chip with Alginate Hydrogel

The curvature array chip was modified by the addition of a perfusion layer comprising perfusion inlets, outlets, and chambers to facilitate the incorporation of an alginate hydrogel. Each cell culture chamber in the array chip had six microfluidic perfusion channels, each measuring 500 μm in width and 120 μm in height and angled at 60°. This configuration ensured that the culture media was distributed evenly through a multi-directional flow towards the culture chamber. To create the hydrogel within the culture chamber, DI water was utilized to fill all channels and chambers. Subsequently, the inlets and outlets of the perfusion chambers were sealed using plugs, and the DI water within the primary culture chamber was carefully removed via aspiration. A 10 μL aliquot of a 1% alginate solution was then introduced to the center of the culture chamber, followed by the addition of 30 μL of a 2 M calcium chloride (CaCl₂) solution to induce crosslinking and facilitate the formation of a 300 μm-thick alginate hydrogel layer. After a 1-minute curing period, any remaining CaCl₂ solution was aspirated, and the hydrogel-embedded chip underwent a sterilization process employing a 70% ethyl alcohol solution and DI water. This prepared the array chip for subsequent cell culture experiments. FIG. 4A-D show representative images of this exemplary embodiment comprising perfusion chambers and hydrogel layers.

1. A cell culture apparatus comprising:
one or more cell culture chambers each comprising a flexible membrane, the flexible membrane separating an upper chamber and a pneumatic chamber and being mechanically integrated with the pneumatic chamber, wherein the pneumatic chamber is fluidly connected to a pneumatic actuator comprising a source of one or more pressurized fluids, the pneumatic actuator being configured to selectively adjust the pressure in the pneumatic chamber, thereby altering the shape of the flexible membrane through mechanical stimulation, and wherein the cell culture apparatus is configured to generate and apply various mechanical stimuli comprising bending stress, shear stress, or a combination thereof to one or more cell types.

2. The apparatus of claim 1, wherein the flexible membrane is comprised of a polymeric material comprising polycarbonate (PC), poly-methyl-meta-acrylate (PMMA), cyclic olefin copolymer (COC), polyimide, polydimethylsiloxane (PDMS), or combinations thereof.

3. The apparatus of claim 2, wherein the flexible membrane is comprised of PDMS.

4. The apparatus of claim 1, wherein the flexible membrane comprises a flat, concave, and/or convex shaped curvature upon mechanical stimulation to modulate bending stress on cells.

5. The apparatus of claim 1, wherein the flexible membrane comprises a uniform thickness across its surface of about 25 μm to about 250 μm.

6. The apparatus of claim 1, wherein each of the one or more cell culture chambers comprises an extracellular matrix layer comprising a hydrogel selected from the group consisting of collagen, elastin, alginate, and combinations thereof disposed on the flexible membrane.

7. The apparatus of claim 6, further comprising one or more perfusion channels, perfusion channel inlets, and perfusion channel outlets each fluidly connected to the hydrogel and configured to deliver one or more liquid fluids to the hydrogel.

8. The apparatus of claim 1, wherein the flexible membrane is comprised of one or more microfluidic layers each independently comprising one or more microfluidic chambers, microfluidic inlets, and microfluidic outlets disposed therein, each of the microfluidic chambers being fluidly connected through one or more porous membranes.

9. The apparatus of claim 8, wherein at least one of the one or more microfluidic chambers further comprises an extracellular matrix layer comprising a hydrogel selected from the group consisting of collagen, elastin, alginate, and combinations thereof.

10. The apparatus of claim 8, further comprising a syringe pump fluidly connected to at least one of the one or more microfluidic inlets or microfluidic outlets, the syringe pump comprising a source of one or more pressurized liquid fluids and configured to modulate shear stress on cells through the microfluidic chambers.

11. The apparatus of claim 10, wherein the syringe pump generates liquid fluid flow rates ranging from about 50 μL/sec to about 150 μL/sec through the microfluidic chambers.

12. The apparatus of claim 1, wherein the one or more pressurized fluids of the pneumatic actuator comprise air, liquid, or a combination thereof.

13. The apparatus of claim 1, wherein the pneumatic actuator generates fluid pressures ranging from about 5 kPa to about 50 kPa through the pneumatic chamber.

14. The apparatus of claim 1, wherein the pneumatic actuator generates fluid pressures at frequencies ranging from about 0.05 Hz to about 5 Hz through the pneumatic chamber.

15. The apparatus of claim 1, wherein the pneumatic chamber is fluidly connected to the pneumatic actuator through an interface component comprising:
one or more interface inlets configured to receive the one or more pressurized fluids from the pneumatic actuator;
one or more interface channels;
one or more interface outlets configured to apply the one or more pressurized fluids to the pneumatic chamber; and
one or more chamber portion outlets.

16. The apparatus of claim 15, wherein the interface component further comprises one or more apertures defining the one or more cell culture chambers.

17. The apparatus of claim 15, further comprising a base component connecting the one or more cell culture chambers to the interface component.

18. The apparatus of claim 1, wherein the apparatus comprises one or more pluralities of cell culture chambers each comprising multiple pneumatic chambers fluidly connected through one or more channels independent from other pluralities of cell culture chambers, and each comprising selectively adjusted pressures generated from the pneumatic actuator independent from other pluralities of cell culture chambers.

19-33. (canceled)

34. A cell culture apparatus comprising:
(a) one or more cell culture chambers each comprising a flexible membrane comprised of one or more microfluidic layers each independently comprising one or more microfluidic chambers, microfluidic inlets, and microfluidic outlets disposed therein, each of the microfluidic chambers being fluidly connected through one or more porous membranes;
(b) an extracellular matrix layer disposed within each of the one or more cell culture chambers, the extracellular matrix layer comprising a hydrogel selected from the group consisting of collagen, elastin, alginate, and combinations thereof;
(c) a means for bonding or injecting the hydrogel to the flexible membrane to form a three-dimensional (3D) cell culture environment;
(d) one or more perfusion channels, perfusion channel inlets, and perfusion channel outlets formed around the hydrogel and configured to deliver one or more cell culture media to the hydrogel;
(e) a means for removing cell culture media from at least one cell culture chamber to enable air exposure, thereby initiating cell differentiation; and
(f) a means for exposing the hydrogel to various biomechanical stimuli.
